SEQUENCE LISTING (627 sequences)

SEQ ID NO: 1 - protein, 16 aa, Homo sapiens
GEKVKIPVAIKPKANK

SEQ ID NO: 2 - protein, 17 aa, Homo sapiens
GEKVKIPVAIKSPKANK

SEQ ID NO: 3 - protein, 15 aa, Homo sapiens
KEYHAEGGKVPIKWM

SEQ ID NO: 4 - protein, 13 aa, Homo sapiens
KIPVAIKHPTSPK

SEQ ID NO: 5 - protein, 13 aa, Homo sapiens
KIPVAIKKPTSPK

SEQ ID NO: 6 - protein, 12 aa, Homo sapiens
KIPVAIKPTSPK

SEQ ID NO: 7 - protein, 13 aa, Homo sapiens
KIPVAIKRPTSPK

SEQ ID NO: 8 - protein, 13 aa, Homo sapiens
MPFGCLLDYVREH

SEQ ID NO: 9 - protein, 18 aa, Homo sapiens
NSTFDSPAHWAQKGSHQI

SEQ ID NO: 10 - protein, 18 aa, Homo sapiens
PREFVENSECIQCHPECL

SEQ ID NO: 11 - protein, 23 aa, Homo sapiens
RGPDNCIQCAHYIDGPHCVKTCP

SEQ ID NO: 12 - protein, 15 aa, Homo sapiens
RMHLPSPTDSNFYRA

SEQ ID NO: 13 - protein, 14 aa, Homo sapiens
SIDDTFLPVPEYIN

SEQ ID NO: 14 - protein, 14 aa, Homo sapiens
VRKCKKCEGPCRKV

SEQ ID NO: 15 - protein, 14 aa, Homo sapiens
VRKSKKSEGPSRKV

SEQ ID NO: 16 - protein, 15 aa, Homo sapiens
ADKDILDEAYVMACA

SEQ ID NO: 17 - protein, 15 aa, Homo sapiens
ADKDILDEAYVMACG

SEQ ID NO: 18 - protein, 15 aa, Homo sapiens
ADKDILDEAYVMACV

SEQ ID NO: 19 - protein, 15 aa, Homo sapiens
ADKDILDEAYVMAIA

SEQ ID NO: 20 - protein, 15 aa, Homo sapiens
ADKDILDEAYVMAIG

SEQ ID NO: 21 - protein, 15 aa, Homo sapiens
ADKDILDEAYVMAIV

SEQ ID NO: 22 - protein, 15 aa, Homo sapiens
ADKDILDEAYVMANA

SEQ ID NO: 23 - protein, 15 aa, Homo sapiens
ADKDILDEAYVMANG

SEQ ID NO: 24 - protein, 15 aa, Homo sapiens
ADKDILDEAYVMANV

SEQ ID NO: 25 - protein, 15 aa, Homo sapiens
ADKDILDEAYVMASA

SEQ ID NO: 26 - protein, 15 aa, Homo sapiens
ADKDILDEAYVMASG

SEQ ID NO: 27 - protein, 15 aa, Homo sapiens
ADKDILDEAYVMASV

SEQ ID NO: 28 - protein, 15 aa, Homo sapiens
ADKDILDEAYVMTCA

SEQ ID NO: 29 - protein, 15 aa, Homo sapiens
ADKDILDEAYVMTCG

SEQ ID NO: 30 - protein, 15 aa, Homo sapiens
ADKDILDEAYVMTCV

SEQ ID NO: 31 - protein, 15 aa, Homo sapiens
ADKDILDEAYVMTIA

SEQ ID NO: 32 - protein, 15 aa, Homo sapiens
ADKDILDEAYVMTIG

SEQ ID NO: 33 - protein, 15 aa, Homo sapiens
ADKDILDEAYVMTIV

SEQ ID NO: 34 - protein, 15 aa, Homo sapiens
ADKDILDEAYVMTNA

SEQ ID NO: 35 - protein, 15 aa, Homo sapiens
ADKDILDEAYVMTNG

SEQ ID NO: 36 - protein, 15 aa, Homo sapiens
ADKDILDEAYVMTNV

SEQ ID NO: 37 - protein, 15 aa, Homo sapiens
ADKDILDEAYVMTSA

SEQ ID NO: 38 - protein, 15 aa, Homo sapiens
ADKDILDEAYVMTSG

SEQ ID NO: 39 - protein, 15 aa, Homo sapiens
ADKDILDEAYVMTSV

SEQ ID NO: 40 - protein, 15 aa, Homo sapiens
ADKEILDEAYVMACA

SEQ ID NO: 41 - protein, 15 aa, Homo sapiens
ADKEILDEAYVMACG

SEQ ID NO: 42 - protein, 15 aa, Homo sapiens
ADKEILDEAYVMACV

SEQ ID NO: 43 - protein, 15 aa, Homo sapiens
ADKEILDEAYVMAIA

SEQ ID NO: 44 - protein, 15 aa, Homo sapiens
ADKEILDEAYVMAIG

SEQ ID NO: 45 - protein, 15 aa, Homo sapiens
ADKEILDEAYVMAIV

SEQ ID NO: 46 - protein, 15 aa, Homo sapiens
ADKEILDEAYVMANA

SEQ ID NO: 47 - protein, 15 aa, Homo sapiens
ADKEILDEAYVMANG

SEQ ID NO: 48 - protein, 15 aa, Homo sapiens
ADKEILDEAYVMANV

SEQ ID NO: 49 - protein, 15 aa, Homo sapiens
ADKEILDEAYVMASA

SEQ ID NO: 50 - protein, 15 aa, Homo sapiens
ADKEILDEAYVMASG

SEQ ID NO: 51 - protein, 15 aa, Homo sapiens
ADKEILDEAYVMASV

SEQ ID NO: 52 - protein, 15 aa, Homo sapiens
ADKEILDEAYVMTCA

SEQ ID NO: 53 - protein, 15 aa, Homo sapiens
ADKEILDEAYVMTCG

SEQ ID NO: 54 - protein, 15 aa, Homo sapiens
ADKEILDEAYVMTCV

SEQ ID NO: 55 - protein, 15 aa, Homo sapiens
ADKEILDEAYVMTIA

SEQ ID NO: 56 - protein, 15 aa, Homo sapiens
ADKEILDEAYVMTIG

SEQ ID NO: 57 - protein, 15 aa, Homo sapiens
ADKEILDEAYVMTIV

SEQ ID NO: 58 - protein, 15 aa, Homo sapiens
ADKEILDEAYVMTNA

SEQ ID NO: 59 - protein, 15 aa, Homo sapiens
ADKEILDEAYVMTNG

SEQ ID NO: 60 - protein, 15 aa, Homo sapiens
ADKEILDEAYVMTNV

SEQ ID NO: 61 - protein, 15 aa, Homo sapiens
ADKEILDEAYVMTSA

SEQ ID NO: 62 - protein, 15 aa, Homo sapiens
ADKEILDEAYVMTSG

SEQ ID NO: 63 - protein, 15 aa, Homo sapiens
ADKEILDEAYVMTSV

SEQ ID NO: 64 - protein, 13 aa, Homo sapiens
AIKELREATSPKA

SEQ ID NO: 65 - protein, 13 aa, Homo sapiens
AIKILREATSPKA

SEQ ID NO: 66 - protein, 13 aa, Homo sapiens
AIKVLREATSPKA

SEQ ID NO: 67 - protein, 15 aa, Homo sapiens
ANKDILDEAYVMACA

SEQ ID NO: 68 - protein, 15 aa, Homo sapiens
ANKDILDEAYVMACG

SEQ ID NO: 69 - protein, 15 aa, Homo sapiens
ANKDILDEAYVMACV

SEQ ID NO: 70 - protein, 15 aa, Homo sapiens
ANKDILDEAYVMAIA

SEQ ID NO: 71 - protein, 15 aa, Homo sapiens
ANKDILDEAYVMAIG

SEQ ID NO: 72 - protein, 15 aa, Homo sapiens
ANKDILDEAYVMAIV

SEQ ID NO: 73 - protein, 15 aa, Homo sapiens
ANKDILDEAYVMANA

SEQ ID NO: 74 - protein, 15 aa, Homo sapiens
ANKDILDEAYVMANG

SEQ ID NO: 75 - protein, 15 aa, Homo sapiens
ANKDILDEAYVMANV

SEQ ID NO: 76 - protein, 15 aa, Homo sapiens
ANKDILDEAYVMASA

SEQ ID NO: 77 - protein, 15 aa, Homo sapiens
ANKDILDEAYVMASG

SEQ ID NO: 78 - protein, 15 aa, Homo sapiens
ANKDILDEAYVMASV

SEQ ID NO: 79 - protein, 15 aa, Homo sapiens
ANKDILDEAYVMTCA

SEQ ID NO: 80 - protein, 15 aa, Homo sapiens
ANKDILDEAYVMTCG

SEQ ID NO: 81 - protein, 15 aa, Homo sapiens
ANKDILDEAYVMTCV

SEQ ID NO: 82 - protein, 15 aa, Homo sapiens
ANKDILDEAYVMTIA

SEQ ID NO: 83 - protein, 15 aa, Homo sapiens
ANKDILDEAYVMTIG

SEQ ID NO: 84 - protein, 15 aa, Homo sapiens
ANKDILDEAYVMTIV

SEQ ID NO: 85 - protein, 15 aa, Homo sapiens
ANKDILDEAYVMTNA

SEQ ID NO: 86 - protein, 15 aa, Homo sapiens
ANKDILDEAYVMTNG

SEQ ID NO: 87 - protein, 15 aa, Homo sapiens
ANKDILDEAYVMTNV

SEQ ID NO: 88 - protein, 15 aa, Homo sapiens
ANKDILDEAYVMTSA

SEQ ID NO: 89 - protein, 15 aa, Homo sapiens
ANKDILDEAYVMTSG

SEQ ID NO: 90 - protein, 15 aa, Homo sapiens
ANKDILDEAYVMTSV

SEQ ID NO: 91 - protein, 15 aa, Homo sapiens
ANKEILDEAYVMACA

SEQ ID NO: 92 - protein, 15 aa, Homo sapiens
ANKEILDEAYVMACG

SEQ ID NO: 93 - protein, 15 aa, Homo sapiens
ANKEILDEAYVMACV

SEQ ID NO: 94 - protein, 15 aa, Homo sapiens
ANKEILDEAYVMAIA

SEQ ID NO: 95 - protein, 15 aa, Homo sapiens
ANKEILDEAYVMAIG

SEQ ID NO: 96 - protein, 15 aa, Homo sapiens
ANKEILDEAYVMAIV

SEQ ID NO: 97 - protein, 15 aa, Homo sapiens
ANKEILDEAYVMANA

SEQ ID NO: 98 - protein, 15 aa, Homo sapiens
ANKEILDEAYVMANG

SEQ ID NO: 99 - protein, 15 aa, Homo sapiens
ANKEILDEAYVMANV

SEQ ID NO: 100 - protein, 15 aa, Homo sapiens
ANKEILDEAYVMASA

SEQ ID NO: 101 - protein, 15 aa, Homo sapiens
ANKEILDEAYVMASG

SEQ ID NO: 102 - protein, 15 aa, Homo sapiens
ANKEILDEAYVMASV

SEQ ID NO: 103 - protein, 15 aa, Homo sapiens
ANKEILDEAYVMTCA

SEQ ID NO: 104 - protein, 15 aa, Homo sapiens
ANKEILDEAYVMTCG

SEQ ID NO: 105 - protein, 15 aa, Homo sapiens
ANKEILDEAYVMTCV

SEQ ID NO: 106 - protein, 15 aa, Homo sapiens
ANKEILDEAYVMTIA

SEQ ID NO: 107 - protein, 15 aa, Homo sapiens
ANKEILDEAYVMTIG

SEQ ID NO: 108 - protein, 15 aa, Homo sapiens
ANKEILDEAYVMTIV

SEQ ID NO: 109 - protein, 15 aa, Homo sapiens
ANKEILDEAYVMTNA

SEQ ID NO: 110 - protein, 15 aa, Homo sapiens
ANKEILDEAYVMTNG

SEQ ID NO: 111 - protein, 15 aa, Homo sapiens
ANKEILDEAYVMTNV

SEQ ID NO: 112 - protein, 15 aa, Homo sapiens
ANKEILDEAYVMTSA

SEQ ID NO: 113 - protein, 15 aa, Homo sapiens
ANKEILDEAYVMTSG

SEQ ID NO: 114 - protein, 15 aa, Homo sapiens
ANKEILDEAYVMTSV

SEQ ID NO: 115 - protein, 15 aa, Homo sapiens
ASKDILDEAYVMACA

SEQ ID NO: 116 - protein, 15 aa, Homo sapiens
ASKDILDEAYVMACG

SEQ ID NO: 117 - protein, 15 aa, Homo sapiens
ASKDILDEAYVMACV

SEQ ID NO: 118 - protein, 15 aa, Homo sapiens
ASKDILDEAYVMAIA

SEQ ID NO: 119 - protein, 15 aa, Homo sapiens
ASKDILDEAYVMAIG

SEQ ID NO: 120 - protein, 15 aa, Homo sapiens
ASKDILDEAYVMAIV

SEQ ID NO: 121 - protein, 15 aa, Homo sapiens
ASKDILDEAYVMANA

SEQ ID NO: 122 - protein, 15 aa, Homo sapiens
ASKDILDEAYVMANG

SEQ ID NO: 123 - protein, 15 aa, Homo sapiens
ASKDILDEAYVMANV

SEQ ID NO: 124 - protein, 15 aa, Homo sapiens
ASKDILDEAYVMASA

SEQ ID NO: 125 - protein, 15 aa, Homo sapiens
ASKDILDEAYVMASG

SEQ ID NO: 126 - protein, 15 aa, Homo sapiens
ASKDILDEAYVMASV

SEQ ID NO: 127 - protein, 15 aa, Homo sapiens
ASKDILDEAYVMTCA

SEQ ID NO: 128 - protein, 15 aa, Homo sapiens
ASKDILDEAYVMTCG

SEQ ID NO: 129 - protein, 15 aa, Homo sapiens
ASKDILDEAYVMTCV

SEQ ID NO: 130 - protein, 15 aa, Homo sapiens
ASKDILDEAYVMTIA

SEQ ID NO: 131 - protein, 15 aa, Homo sapiens
ASKDILDEAYVMTIG

SEQ ID NO: 132 - protein, 15 aa, Homo sapiens
ASKDILDEAYVMTIV

SEQ ID NO: 133 - protein, 15 aa, Homo sapiens
ASKDILDEAYVMTNA

SEQ ID NO: 134 - protein, 15 aa, Homo sapiens
ASKDILDEAYVMTNG

SEQ ID NO: 135 - protein, 15 aa, Homo sapiens
ASKDILDEAYVMTNV

SEQ ID NO: 136 - protein, 15 aa, Homo sapiens
ASKDILDEAYVMTSA

SEQ ID NO: 137 - protein, 15 aa, Homo sapiens
ASKDILDEAYVMTSG

SEQ ID NO: 138 - protein, 15 aa, Homo sapiens
ASKDILDEAYVMTSV

SEQ ID NO: 139 - protein, 15 aa, Homo sapiens
ASKEILDEAYVMACA

SEQ ID NO: 140 - protein, 15 aa, Homo sapiens
ASKEILDEAYVMACG

SEQ ID NO: 141 - protein, 15 aa, Homo sapiens
ASKEILDEAYVMACV

SEQ ID NO: 142 - protein, 15 aa, Homo sapiens
ASKEILDEAYVMAIA

SEQ ID NO: 143 - protein, 15 aa, Homo sapiens
ASKEILDEAYVMAIG

SEQ ID NO: 144 - protein, 15 aa, Homo sapiens
ASKEILDEAYVMAIV

SEQ ID NO: 145 - protein, 15 aa, Homo sapiens
ASKEILDEAYVMANA

SEQ ID NO: 146 - protein, 15 aa, Homo sapiens
ASKEILDEAYVMANG

SEQ ID NO: 147 - protein, 15 aa, Homo sapiens
ASKEILDEAYVMANV

SEQ ID NO: 148 - protein, 15 aa, Homo sapiens
ASKEILDEAYVMASA

SEQ ID NO: 149 - protein, 15 aa, Homo sapiens
ASKEILDEAYVMASG

SEQ ID NO: 150 - protein, 15 aa, Homo sapiens
ASKEILDEAYVMASV

SEQ ID NO: 151 - protein, 15 aa, Homo sapiens
ASKEILDEAYVMTCA

SEQ ID NO: 152 - protein, 15 aa, Homo sapiens
ASKEILDEAYVMTCG

SEQ ID NO: 153 - protein, 15 aa, Homo sapiens
ASKEILDEAYVMTCV

SEQ ID NO: 154 - protein, 15 aa, Homo sapiens
ASKEILDEAYVMTIA

SEQ ID NO: 155 - protein, 15 aa, Homo sapiens
ASKEILDEAYVMTIG

SEQ ID NO: 156 - protein, 15 aa, Homo sapiens
ASKEILDEAYVMTIV

SEQ ID NO: 157 - protein, 15 aa, Homo sapiens
ASKEILDEAYVMTNA

SEQ ID NO: 158 - protein, 15 aa, Homo sapiens
ASKEILDEAYVMTNG

SEQ ID NO: 159 - protein, 15 aa, Homo sapiens
ASKEILDEAYVMTNV

SEQ ID NO: 160 - protein, 15 aa, Homo sapiens
ASKEILDEAYVMTSA

SEQ ID NO: 161 - protein, 15 aa, Homo sapiens
ASKEILDEAYVMTSG

SEQ ID NO: 162 - protein, 15 aa, Homo sapiens
ASKEILDEAYVMTSV

SEQ ID NO: 163 - protein, 13 aa, Homo sapiens
CLLVHRDLAARNV

SEQ ID NO: 164 - protein, 13 aa, Homo sapiens
CRLVHRDLAARNV

SEQ ID NO: 165 - protein, 15 aa, Homo sapiens
DDKDILDEAYVMACA

SEQ ID NO: 166 - protein, 15 aa, Homo sapiens
DDKDILDEAYVMACG

SEQ ID NO: 167 - protein, 15 aa, Homo sapiens
DDKDILDEAYVMACV

SEQ ID NO: 168 - protein, 15 aa, Homo sapiens
DDKDILDEAYVMAIA

SEQ ID NO: 169 - protein, 15 aa, Homo sapiens
DDKDILDEAYVMAIG

SEQ ID NO: 170 - protein, 15 aa, Homo sapiens
DDKDILDEAYVMAIV

SEQ ID NO: 171 - protein, 15 aa, Homo sapiens
DDKDILDEAYVMANA

SEQ ID NO: 172 - protein, 15 aa, Homo sapiens
DDKDILDEAYVMANG

SEQ ID NO: 173 - protein, 15 aa, Homo sapiens
DDKDILDEAYVMANV

SEQ ID NO: 174 - protein, 15 aa, Homo sapiens
DDKDILDEAYVMASA

SEQ ID NO: 175 - protein, 15 aa, Homo sapiens
DDKDILDEAYVMASG

SEQ ID NO: 176 - protein, 15 aa, Homo sapiens
DDKDILDEAYVMASV

SEQ ID NO: 177 - protein, 15 aa, Homo sapiens
DDKDILDEAYVMTCA

SEQ ID NO: 178 - protein, 15 aa, Homo sapiens
DDKDILDEAYVMTCG

SEQ ID NO: 179 - protein, 15 aa, Homo sapiens
DDKDILDEAYVMTCV

SEQ ID NO: 180 - protein, 15 aa, Homo sapiens
DDKDILDEAYVMTIA

SEQ ID NO: 181 - protein, 15 aa, Homo sapiens
DDKDILDEAYVMTIG

SEQ ID NO: 182 - protein, 15 aa, Homo sapiens
DDKDILDEAYVMTIV

SEQ ID NO: 183 - protein, 15 aa, Homo sapiens
DDKDILDEAYVMTNA

SEQ ID NO: 184 - protein, 15 aa, Homo sapiens
DDKDILDEAYVMTNG

SEQ ID NO: 185 - protein, 15 aa, Homo sapiens
DDKDILDEAYVMTNV

SEQ ID NO: 186 - protein, 15 aa, Homo sapiens
DDKDILDEAYVMTSA

SEQ ID NO: 187 - protein, 15 aa, Homo sapiens
DDKDILDEAYVMTSG

SEQ ID NO: 188 - protein, 15 aa, Homo sapiens
DDKDILDEAYVMTSV

SEQ ID NO: 189 - protein, 15 aa, Homo sapiens
DDKEILDEAYVMACA

SEQ ID NO: 190 - protein, 15 aa, Homo sapiens
DDKEILDEAYVMACG

SEQ ID NO: 191 - protein, 15 aa, Homo sapiens
DDKEILDEAYVMACV

SEQ ID NO: 192 - protein, 15 aa, Homo sapiens
DDKEILDEAYVMAIA

SEQ ID NO: 193 - protein, 15 aa, Homo sapiens
DDKEILDEAYVMAIG

SEQ ID NO: 194 - protein, 15 aa, Homo sapiens
DDKEILDEAYVMAIV

SEQ ID NO: 195 - protein, 15 aa, Homo sapiens
DDKEILDEAYVMANA

SEQ ID NO: 196 - protein, 15 aa, Homo sapiens
DDKEILDEAYVMANG

SEQ ID NO: 197 - protein, 15 aa, Homo sapiens
DDKEILDEAYVMANV

SEQ ID NO: 198 - protein, 15 aa, Homo sapiens
DDKEILDEAYVMASA

SEQ ID NO: 199 - protein, 15 aa, Homo sapiens
DDKEILDEAYVMASG

SEQ ID NO: 200 - protein, 15 aa, Homo sapiens
DDKEILDEAYVMASV

SEQ ID NO: 201 - protein, 15 aa, Homo sapiens
DDKEILDEAYVMTCA

SEQ ID NO: 202 - protein, 15 aa, Homo sapiens
DDKEILDEAYVMTCG

SEQ ID NO: 203 - protein, 15 aa, Homo sapiens
DDKEILDEAYVMTCV

SEQ ID NO: 204 - protein, 15 aa, Homo sapiens
DDKEILDEAYVMTIA

SEQ ID NO: 205 - protein, 15 aa, Homo sapiens
DDKEILDEAYVMTIG

SEQ ID NO: 206 - protein, 15 aa, Homo sapiens
DDKEILDEAYVMTIV

SEQ ID NO: 207 - protein, 15 aa, Homo sapiens
DDKEILDEAYVMTNA

SEQ ID NO: 208 - protein, 15 aa, Homo sapiens
DDKEILDEAYVMTNG

SEQ ID NO: 209 - protein, 15 aa, Homo sapiens
DDKEILDEAYVMTNV

SEQ ID NO: 210 - protein, 15 aa, Homo sapiens
DDKEILDEAYVMTSA

SEQ ID NO: 211 - protein, 15 aa, Homo sapiens
DDKEILDEAYVMTSG

SEQ ID NO: 212 - protein, 15 aa, Homo sapiens
DDKEILDEAYVMTSV

SEQ ID NO: 213 - protein, 15 aa, Homo sapiens
DNKDILDEAYVMACA

SEQ ID NO: 214 - protein, 15 aa, Homo sapiens
DNKDILDEAYVMACG

SEQ ID NO: 215 - protein, 15 aa, Homo sapiens
DNKDILDEAYVMACV

SEQ ID NO: 216 - protein, 15 aa, Homo sapiens
DNKDILDEAYVMAIA

SEQ ID NO: 217 - protein, 15 aa, Homo sapiens
DNKDILDEAYVMAIG

SEQ ID NO: 218 - protein, 15 aa, Homo sapiens
DNKDILDEAYVMAIV

SEQ ID NO: 219 - protein, 15 aa, Homo sapiens
DNKDILDEAYVMANA

SEQ ID NO: 220 - protein, 15 aa, Homo sapiens
DNKDILDEAYVMANG

SEQ ID NO: 221 - protein, 15 aa, Homo sapiens
DNKDILDEAYVMANV

SEQ ID NO: 222 - protein, 15 aa, Homo sapiens
DNKDILDEAYVMASA

SEQ ID NO: 223 - protein, 15 aa, Homo sapiens
DNKDILDEAYVMASG

SEQ ID NO: 224 - protein, 15 aa, Homo sapiens
DNKDILDEAYVMASV

SEQ ID NO: 225 - protein, 15 aa, Homo sapiens
DNKDILDEAYVMTCA

SEQ ID NO: 226 - protein, 15 aa, Homo sapiens
DNKDILDEAYVMTCG

SEQ ID NO: 227 - protein, 15 aa, Homo sapiens
DNKDILDEAYVMTCV

SEQ ID NO: 228 - protein, 15 aa, Homo sapiens
DNKDILDEAYVMTIA

SEQ ID NO: 229 - protein, 15 aa, Homo sapiens
DNKDILDEAYVMTIG

SEQ ID NO: 230 - protein, 15 aa, Homo sapiens
DNKDILDEAYVMTIV

SEQ ID NO: 231 - protein, 15 aa, Homo sapiens
DNKDILDEAYVMTNA

SEQ ID NO: 232 - protein, 15 aa, Homo sapiens
DNKDILDEAYVMTNG

SEQ ID NO: 233 - protein, 15 aa, Homo sapiens
DNKDILDEAYVMTNV

SEQ ID NO: 234 - protein, 15 aa, Homo sapiens
DNKDILDEAYVMTSA

SEQ ID NO: 235 - protein, 15 aa, Homo sapiens
DNKDILDEAYVMTSG

SEQ ID NO: 236 - protein, 15 aa, Homo sapiens
DNKDILDEAYVMTSV

SEQ ID NO: 237 - protein, 15 aa, Homo sapiens
DNKEILDEAYVMACA

SEQ ID NO: 238 - protein, 15 aa, Homo sapiens
DNKEILDEAYVMACG

SEQ ID NO: 239 - protein, 15 aa, Homo sapiens
DNKEILDEAYVMACV

SEQ ID NO: 240 - protein, 15 aa, Homo sapiens
DNKEILDEAYVMAIA

SEQ ID NO: 241 - protein, 15 aa, Homo sapiens
DNKEILDEAYVMAIG

SEQ ID NO: 242 - protein, 15 aa, Homo sapiens
DNKEILDEAYVMAIV

SEQ ID NO: 243 - protein, 15 aa, Homo sapiens
DNKEILDEAYVMANA

SEQ ID NO: 244 - protein, 15 aa, Homo sapiens
DNKEILDEAYVMANG

SEQ ID NO: 245 - protein, 15 aa, Homo sapiens
DNKEILDEAYVMANV

SEQ ID NO: 246 - protein, 15 aa, Homo sapiens
DNKEILDEAYVMASA

SEQ ID NO: 247 - protein, 15 aa, Homo sapiens
DNKEILDEAYVMASG

SEQ ID NO: 248 - protein, 15 aa, Homo sapiens
DNKEILDEAYVMASV

SEQ ID NO: 249 - protein, 15 aa, Homo sapiens
DNKEILDEAYVMTCA

SEQ ID NO: 250 - protein, 15 aa, Homo sapiens
DNKEILDEAYVMTCG

SEQ ID NO: 251 - protein, 15 aa, Homo sapiens
DNKEILDEAYVMTCV

SEQ ID NO: 252 - protein, 15 aa, Homo sapiens
DNKEILDEAYVMTIA

SEQ ID NO: 253 - protein, 15 aa, Homo sapiens
DNKEILDEAYVMTIG

SEQ ID NO: 254 - protein, 15 aa, Homo sapiens
DNKEILDEAYVMTIV

SEQ ID NO: 255 - protein, 15 aa, Homo sapiens
DNKEILDEAYVMTNA

SEQ ID NO: 256 - protein, 15 aa, Homo sapiens
DNKEILDEAYVMTNG

SEQ ID NO: 257 - protein, 15 aa, Homo sapiens
DNKEILDEAYVMTNV

SEQ ID NO: 258 - protein, 15 aa, Homo sapiens
DNKEILDEAYVMTSA

SEQ ID NO: 259 - protein, 15 aa, Homo sapiens
DNKEILDEAYVMTSG

SEQ ID NO: 260 - protein, 15 aa, Homo sapiens
DNKEILDEAYVMTSV

SEQ ID NO: 261 - protein, 15 aa, Homo sapiens
DSKDILDEAYVMACA

SEQ ID NO: 262 - protein, 15 aa, Homo sapiens
DSKDILDEAYVMACG

SEQ ID NO: 263 - protein, 15 aa, Homo sapiens
DSKDILDEAYVMACV

SEQ ID NO: 264 - protein, 15 aa, Homo sapiens
DSKDILDEAYVMAIA

SEQ ID NO: 265 - protein, 15 aa, Homo sapiens
DSKDILDEAYVMAIG

SEQ ID NO: 266 - protein, 15 aa, Homo sapiens
DSKDILDEAYVMAIV

SEQ ID NO: 267 - protein, 15 aa, Homo sapiens
DSKDILDEAYVMANA

SEQ ID NO: 268 - protein, 15 aa, Homo sapiens
DSKDILDEAYVMANG

SEQ ID NO: 269 - protein, 15 aa, Homo sapiens
DSKDILDEAYVMANV

SEQ ID NO: 270 - protein, 15 aa, Homo sapiens
DSKDILDEAYVMASA

SEQ ID NO: 271 - protein, 15 aa, Homo sapiens
DSKDILDEAYVMASG

SEQ ID NO: 272 - protein, 15 aa, Homo sapiens
DSKDILDEAYVMASV

SEQ ID NO: 273 - protein, 15 aa, Homo sapiens
DSKDILDEAYVMTCA

SEQ ID NO: 274 - protein, 15 aa, Homo sapiens
DSKDILDEAYVMTCG

SEQ ID NO: 275 - protein, 15 aa, Homo sapiens
DSKDILDEAYVMTCV

SEQ ID NO: 276 - protein, 15 aa, Homo sapiens
DSKDILDEAYVMTIA

SEQ ID NO: 277 - protein, 15 aa, Homo sapiens
DSKDILDEAYVMTIG

SEQ ID NO: 278 - protein, 15 aa, Homo sapiens
DSKDILDEAYVMTIV

SEQ ID NO: 279 - protein, 15 aa, Homo sapiens
DSKDILDEAYVMTNA

SEQ ID NO: 280 - protein, 15 aa, Homo sapiens
DSKDILDEAYVMTNG

SEQ ID NO: 281 - protein, 15 aa, Homo sapiens
DSKDILDEAYVMTNV

SEQ ID NO: 282 - protein, 15 aa, Homo sapiens
DSKDILDEAYVMTSA

SEQ ID NO: 283 - protein, 15 aa, Homo sapiens
DSKDILDEAYVMTSG

SEQ ID NO: 284 - protein, 15 aa, Homo sapiens
DSKDILDEAYVMTSV

SEQ ID NO: 285 - protein, 15 aa, Homo sapiens
DSKEILDEAYVMACA

SEQ ID NO: 286 - protein, 15 aa, Homo sapiens
DSKEILDEAYVMACG

SEQ ID NO: 287 - protein, 15 aa, Homo sapiens
DSKEILDEAYVMACV

SEQ ID NO: 288 - protein, 15 aa, Homo sapiens
DSKEILDEAYVMAIA

SEQ ID NO: 289 - protein, 15 aa, Homo sapiens
DSKEILDEAYVMAIG

SEQ ID NO: 290 - protein, 15 aa, Homo sapiens
DSKEILDEAYVMAIV

SEQ ID NO: 291 - protein, 15 aa, Homo sapiens
DSKEILDEAYVMANA

SEQ ID NO: 292 - protein, 15 aa, Homo sapiens
DSKEILDEAYVMANG

SEQ ID NO: 293 - protein, 15 aa, Homo sapiens
DSKEILDEAYVMANV

SEQ ID NO: 294 - protein, 15 aa, Homo sapiens
DSKEILDEAYVMASA

SEQ ID NO: 295 - protein, 15 aa, Homo sapiens
DSKEILDEAYVMASG

SEQ ID NO: 296 - protein, 15 aa, Homo sapiens
DSKEILDEAYVMASV

SEQ ID NO: 297 - protein, 15 aa, Homo sapiens
DSKEILDEAYVMTCA

SEQ ID NO: 298 - protein, 15 aa, Homo sapiens
DSKEILDEAYVMTCG

SEQ ID NO: 299 - protein, 15 aa, Homo sapiens
DSKEILDEAYVMTCV

SEQ ID NO: 300 - protein, 15 aa, Homo sapiens
DSKEILDEAYVMTIA

SEQ ID NO: 301 - protein, 15 aa, Homo sapiens
DSKEILDEAYVMTIG

SEQ ID NO: 302 - protein, 15 aa, Homo sapiens
DSKEILDEAYVMTIV

SEQ ID NO: 303 - protein, 15 aa, Homo sapiens
DSKEILDEAYVMTNA

SEQ ID NO: 304 - protein, 15 aa, Homo sapiens
DSKEILDEAYVMTNG

SEQ ID NO: 305 - protein, 15 aa, Homo sapiens
DSKEILDEAYVMTNV

SEQ ID NO: 306 - protein, 15 aa, Homo sapiens
DSKEILDEAYVMTSA

SEQ ID NO: 307 - protein, 15 aa, Homo sapiens
DSKEILDEAYVMTSG

SEQ ID NO: 308 - protein, 15 aa, Homo sapiens
DSKEILDEAYVMTSV

SEQ ID NO: 309 - protein, 15 aa, Homo sapiens
HYQDPHSTAVGNPEY

SEQ ID NO: 310 - protein, 14 aa, Homo sapiens
KVKIPVAIAPSPKA

SEQ ID NO: 311 - protein, 14 aa, Homo sapiens
KVKIPVAIATSPKA

SEQ ID NO: 312 - protein, 15 aa, Homo sapiens
KVKIPVAIEAPSPKA

SEQ ID NO: 313 - protein, 15 aa, Homo sapiens
KVKIPVAIEATSPKA

SEQ ID NO: 314 - protein, 16 aa, Homo sapiens
KVKIPVAIEEAPSPKA

SEQ ID NO: 315 - protein, 16 aa, Homo sapiens
KVKIPVAIEEATSPKA

SEQ ID NO: 316 - protein, 15 aa, Homo sapiens
KVKIPVAIEEPSPKA

SEQ ID NO: 317 - protein, 15 aa, Homo sapiens
KVKIPVAIEETSPKA

SEQ ID NO: 318 - protein, 14 aa, Homo sapiens
KVKIPVAIEPSPKA

SEQ ID NO: 319 - protein, 16 aa, Homo sapiens
KVKIPVAIERAPSPKA

SEQ ID NO: 320 - protein, 16 aa, Homo sapiens
KVKIPVAIERATSPKA

SEQ ID NO: 321 - protein, 17 aa, Homo sapiens
KVKIPVAIEREAPSPKA

SEQ ID NO: 322 - protein, 17 aa, Homo sapiens
KVKIPVAIEREATSPKA

SEQ ID NO: 323 - protein, 16 aa, Homo sapiens
KVKIPVAIEREPSPKA

SEQ ID NO: 324 - protein, 16 aa, Homo sapiens
KVKIPVAIERETSPKA

SEQ ID NO: 325 - protein, 15 aa, Homo sapiens
KVKIPVAIERPSPKA

SEQ ID NO: 326 - protein, 15 aa, Homo sapiens
KVKIPVAIERTSPKA

SEQ ID NO: 327 - protein, 14 aa, Homo sapiens
KVKIPVAIETSPKA

SEQ ID NO: 328 - protein, 15 aa, Homo sapiens
KVKIPVAIKAPSPKA

SEQ ID NO: 329 - protein, 15 aa, Homo sapiens
KVKIPVAIKATSPKA

SEQ ID NO: 330 - protein, 16 aa, Homo sapiens
KVKIPVAIKEAPSPKA

SEQ ID NO: 331 - protein, 16 aa, Homo sapiens
KVKIPVAIKEATSPKA

SEQ ID NO: 332 - protein, 17 aa, Homo sapiens
KVKIPVAIKEEAPSPKA

SEQ ID NO: 333 - protein, 17 aa, Homo sapiens
KVKIPVAIKEEATSPKA

SEQ ID NO: 334 - protein, 16 aa, Homo sapiens
KVKIPVAIKEEPSPKA

SEQ ID NO: 335 - protein, 16 aa, Homo sapiens
KVKIPVAIKEETSPKA

SEQ ID NO: 336 - protein, 15 aa, Homo sapiens
KVKIPVAIKEPSPKA

SEQ ID NO: 337 - protein, 17 aa, Homo sapiens
KVKIPVAIKERAPSPKA

SEQ ID NO: 338 - protein, 17 aa, Homo sapiens
KVKIPVAIKERATSPKA

SEQ ID NO: 339 - protein, 18 aa, Homo sapiens
KVKIPVAIKEREAPSPKA

SEQ ID NO: 340 - protein, 18 aa, Homo sapiens
KVKIPVAIKEREATSPKA

SEQ ID NO: 341 - protein, 17 aa, Homo sapiens
KVKIPVAIKEREPSPKA

SEQ ID NO: 342 - protein, 17 aa, Homo sapiens
KVKIPVAIKERETSPKA

SEQ ID NO: 343 - protein, 16 aa, Homo sapiens
KVKIPVAIKERPSPKA

SEQ ID NO: 344 - protein, 16 aa, Homo sapiens
KVKIPVAIKERTSPKA

SEQ ID NO: 345 - protein, 15 aa, Homo sapiens
KVKIPVAIKETSPKA

SEQ ID NO: 346 - protein, 14 aa, Homo sapiens
KVKIPVAIKPSPKA

SEQ ID NO: 347 - protein, 16 aa, Homo sapiens
KVKIPVAIKRAPSPKA

SEQ ID NO: 348 - protein, 16 aa, Homo sapiens
KVKIPVAIKRATSPKA

SEQ ID NO: 349 - protein, 17 aa, Homo sapiens
KVKIPVAIKREAPSPKA

SEQ ID NO: 350 - protein, 17 aa, Homo sapiens
KVKIPVAIKREATSPKA

SEQ ID NO: 351 - protein, 16 aa, Homo sapiens
KVKIPVAIKREPSPKA

SEQ ID NO: 352 - protein, 16 aa, Homo sapiens
KVKIPVAIKRETSPKA

SEQ ID NO: 353 - protein, 15 aa, Homo sapiens
KVKIPVAIKRPSPKA

SEQ ID NO: 354 - protein, 15 aa, Homo sapiens
KVKIPVAIKRTSPKA

SEQ ID NO: 355 - protein, 14 aa, Homo sapiens
KVKIPVAIKTSPKA

SEQ ID NO: 356 - protein, 13 aa, Homo sapiens
KVKIPVAIPSPKA

SEQ ID NO: 357 - protein, 15 aa, Homo sapiens
KVKIPVAIRAPSPKA

SEQ ID NO: 358 - protein, 15 aa, Homo sapiens
KVKIPVAIRATSPKA

SEQ ID NO: 359 - protein, 16 aa, Homo sapiens
KVKIPVAIREAPSPKA

SEQ ID NO: 360 - protein, 16 aa, Homo sapiens
KVKIPVAIREATSPKA

SEQ ID NO: 361 - protein, 15 aa, Homo sapiens
KVKIPVAIREPSPKA

SEQ ID NO: 362 - protein, 15 aa, Homo sapiens
KVKIPVAIRETSPKA

SEQ ID NO: 363 - protein, 14 aa, Homo sapiens
KVKIPVAIRPSPKA

SEQ ID NO: 364 - protein, 14 aa, Homo sapiens
KVKIPVAIRTSPKA

SEQ ID NO: 365 - protein, 13 aa, Homo sapiens
KVKIPVAITSPKA

SEQ ID NO: 366 - protein, 13 aa, Homo sapiens
LREEILDEAYVMA

SEQ ID NO: 367 - protein, 13 aa, Homo sapiens
LREPILDEAYVMA

SEQ ID NO: 368 - protein, 18 aa, Homo sapiens
PEGEKAKIPVAIKELREA

SEQ ID NO: 369 - protein, 18 aa, Homo sapiens
PEGEKAKIPVAIRELREA

SEQ ID NO: 370 - protein, 18 aa, Homo sapiens
PEGEKVKIPVAIKELREA

SEQ ID NO: 371 - protein, 18 aa, Homo sapiens
PEGEKVKIPVAIRELREA

SEQ ID NO: 372 - protein, 13 aa, Homo sapiens
RLLVHRDLAARNV

SEQ ID NO: 373 - protein, 13 aa, Homo sapiens
RRLVHRDLAARNV

SEQ ID NO: 374 - protein, 15 aa, Homo sapiens
SDKDILDEAYVMACA

SEQ ID NO: 375 - protein, 15 aa, Homo sapiens
SDKDILDEAYVMACG

SEQ ID NO: 376 - protein, 15 aa, Homo sapiens
SDKDILDEAYVMACV

SEQ ID NO: 377 - protein, 15 aa, Homo sapiens
SDKDILDEAYVMAIA

SEQ ID NO: 378 - protein, 15 aa, Homo sapiens
SDKDILDEAYVMAIG

SEQ ID NO: 379 - protein, 15 aa, Homo sapiens
SDKDILDEAYVMAIV

SEQ ID NO: 380 - protein, 15 aa, Homo sapiens
SDKDILDEAYVMANA

SEQ ID NO: 381 - protein, 15 aa, Homo sapiens
SDKDILDEAYVMANG

SEQ ID NO: 382 - protein, 15 aa, Homo sapiens
SDKDILDEAYVMANV

SEQ ID NO: 383 - protein, 15 aa, Homo sapiens
SDKDILDEAYVMASA

SEQ ID NO: 384 - protein, 15 aa, Homo sapiens
SDKDILDEAYVMASG

SEQ ID NO: 385 - protein, 15 aa, Homo sapiens
SDKDILDEAYVMASV

SEQ ID NO: 386 - protein, 15 aa, Homo sapiens
SDKDILDEAYVMTCA

SEQ ID NO: 387 - protein, 15 aa, Homo sapiens
SDKDILDEAYVMTCG

SEQ ID NO: 388 - protein, 15 aa, Homo sapiens
SDKDILDEAYVMTCV

SEQ ID NO: 389 - protein, 15 aa, Homo sapiens
SDKDILDEAYVMTIA

SEQ ID NO: 390 - protein, 15 aa, Homo sapiens
SDKDILDEAYVMTIG

SEQ ID NO: 391 - protein, 15 aa, Homo sapiens
SDKDILDEAYVMTIV

SEQ ID NO: 392 - protein, 15 aa, Homo sapiens
SDKDILDEAYVMTNA

SEQ ID NO: 393 - protein, 15 aa, Homo sapiens
SDKDILDEAYVMTNG

SEQ ID NO: 394 - protein, 15 aa, Homo sapiens
SDKDILDEAYVMTNV

SEQ ID NO: 395 - protein, 15 aa, Homo sapiens
SDKDILDEAYVMTSA

SEQ ID NO: 396 - protein, 15 aa, Homo sapiens
SDKDILDEAYVMTSG

SEQ ID NO: 397 - protein, 15 aa, Homo sapiens
SDKDILDEAYVMTSV

SEQ ID NO: 398 - protein, 15 aa, Homo sapiens
SDKEILDEAYVMACA

SEQ ID NO: 399 - protein, 15 aa, Homo sapiens
SDKEILDEAYVMACG

SEQ ID NO: 400 - protein, 15 aa, Homo sapiens
SDKEILDEAYVMACV

SEQ ID NO: 401 - protein, 15 aa, Homo sapiens
SDKEILDEAYVMAIA

SEQ ID NO: 402 - protein, 15 aa, Homo sapiens
SDKEILDEAYVMAIG

SEQ ID NO: 403 - protein, 15 aa, Homo sapiens
SDKEILDEAYVMAIV

SEQ ID NO: 404 - protein, 15 aa, Homo sapiens
SDKEILDEAYVMANA

SEQ ID NO: 405 - protein, 15 aa, Homo sapiens
SDKEILDEAYVMANG

SEQ ID NO: 406 - protein, 15 aa, Homo sapiens
SDKEILDEAYVMANV

SEQ ID NO: 407 - protein, 15 aa, Homo sapiens
SDKEILDEAYVMASA

SEQ ID NO: 408 - protein, 15 aa, Homo sapiens
SDKEILDEAYVMASG

SEQ ID NO: 409 - protein, 15 aa, Homo sapiens
SDKEILDEAYVMASV

SEQ ID NO: 410 - protein, 15 aa, Homo sapiens
SDKEILDEAYVMTCA

SEQ ID NO: 411 - protein, 15 aa, Homo sapiens
SDKEILDEAYVMTCG

SEQ ID NO: 412 - protein, 15 aa, Homo sapiens
SDKEILDEAYVMTCV

SEQ ID NO: 413 - protein, 15 aa, Homo sapiens
SDKEILDEAYVMTIA

SEQ ID NO: 414 - protein, 15 aa, Homo sapiens
SDKEILDEAYVMTIG

SEQ ID NO: 415 - protein, 15 aa, Homo sapiens
SDKEILDEAYVMTIV

SEQ ID NO: 416 - protein, 15 aa, Homo sapiens
SDKEILDEAYVMTNA

SEQ ID NO: 417 - protein, 15 aa, Homo sapiens
SDKEILDEAYVMTNG

SEQ ID NO: 418 - protein, 15 aa, Homo sapiens
SDKEILDEAYVMTNV

SEQ ID NO: 419 - protein, 15 aa, Homo sapiens
SDKEILDEAYVMTSA

SEQ ID NO: 420 - protein, 15 aa, Homo sapiens
SDKEILDEAYVMTSG

SEQ ID NO: 421 - protein, 15 aa, Homo sapiens
SDKEILDEAYVMTSV

SEQ ID NO: 422 - protein, 15 aa, Homo sapiens
SNKDILDEAYVMACA

SEQ ID NO: 423 - protein, 15 aa, Homo sapiens
SNKDILDEAYVMACG

SEQ ID NO: 424 - protein, 15 aa, Homo sapiens
SNKDILDEAYVMACV

SEQ ID NO: 425 - protein, 15 aa, Homo sapiens
SNKDILDEAYVMAIA

SEQ ID NO: 426 - protein, 15 aa, Homo sapiens
SNKDILDEAYVMAIG

SEQ ID NO: 427 - protein, 15 aa, Homo sapiens
SNKDILDEAYVMAIV

SEQ ID NO: 428 - protein, 15 aa, Homo sapiens
SNKDILDEAYVMANA

SEQ ID NO: 429 - protein, 15 aa, Homo sapiens
SNKDILDEAYVMANG

SEQ ID NO: 430 - protein, 15 aa, Homo sapiens
SNKDILDEAYVMANV

SEQ ID NO: 431 - protein, 15 aa, Homo sapiens
SNKDILDEAYVMASA

SEQ ID NO: 432 - protein, 15 aa, Homo sapiens
SNKDILDEAYVMASG

SEQ ID NO: 433 - protein, 15 aa, Homo sapiens
SNKDILDEAYVMASV

SEQ ID NO: 434 - protein, 15 aa, Homo sapiens
SNKDILDEAYVMTCA

SEQ ID NO: 435 - protein, 15 aa, Homo sapiens
SNKDILDEAYVMTCG

SEQ ID NO: 436 - protein, 15 aa, Homo sapiens
SNKDILDEAYVMTCV

SEQ ID NO: 437 - protein, 15 aa, Homo sapiens
SNKDILDEAYVMTIA

SEQ ID NO: 438 - protein, 15 aa, Homo sapiens
SNKDILDEAYVMTIG

SEQ ID NO: 439 - protein, 15 aa, Homo sapiens
SNKDILDEAYVMTIV

SEQ ID NO: 440 - protein, 15 aa, Homo sapiens
SNKDILDEAYVMTNA

SEQ ID NO: 441 - protein, 15 aa, Homo sapiens
SNKDILDEAYVMTNG

SEQ ID NO: 442 - protein, 15 aa, Homo sapiens
SNKDILDEAYVMTNV

SEQ ID NO: 443 - protein, 15 aa, Homo sapiens
SNKDILDEAYVMTSA

SEQ ID NO: 444 - protein, 15 aa, Homo sapiens
SNKDILDEAYVMTSG

SEQ ID NO: 445 - protein, 15 aa, Homo sapiens
SNKDILDEAYVMTSV

SEQ ID NO: 446 - protein, 15 aa, Homo sapiens
SNKEILDEAYVMACA

SEQ ID NO: 447 - protein, 15 aa, Homo sapiens
SNKEILDEAYVMACG

SEQ ID NO: 448 - protein, 15 aa, Homo sapiens
SNKEILDEAYVMACV

SEQ ID NO: 449 - protein, 15 aa, Homo sapiens
SNKEILDEAYVMAIA

SEQ ID NO: 450 - protein, 15 aa, Homo sapiens
SNKEILDEAYVMAIG

SEQ ID NO: 451 - protein, 15 aa, Homo sapiens
SNKEILDEAYVMAIV

SEQ ID NO: 452 - protein, 15 aa, Homo sapiens
SNKEILDEAYVMANA

SEQ ID NO: 453 - protein, 15 aa, Homo sapiens
SNKEILDEAYVMANG

SEQ ID NO: 454 - protein, 15 aa, Homo sapiens
SNKEILDEAYVMANV

SEQ ID NO: 455 - protein, 15 aa, Homo sapiens
SNKEILDEAYVMASA

SEQ ID NO: 456 - protein, 15 aa, Homo sapiens
SNKEILDEAYVMASG

SEQ ID NO: 457 - protein, 15 aa, Homo sapiens
SNKEILDEAYVMASV

SEQ ID NO: 458 - protein, 15 aa, Homo sapiens
SNKEILDEAYVMTCA

SEQ ID NO: 459 - protein, 15 aa, Homo sapiens
SNKEILDEAYVMTCG

SEQ ID NO: 460 - protein, 15 aa, Homo sapiens
SNKEILDEAYVMTCV

SEQ ID NO: 461 - protein, 15 aa, Homo sapiens
SNKEILDEAYVMTIA

SEQ ID NO: 462 - protein, 15 aa, Homo sapiens
SNKEILDEAYVMTIG

SEQ ID NO: 463 - protein, 15 aa, Homo sapiens
SNKEILDEAYVMTIV

SEQ ID NO: 464 - protein, 15 aa, Homo sapiens
SNKEILDEAYVMTNA

SEQ ID NO: 465 - protein, 15 aa, Homo sapiens
SNKEILDEAYVMTNG

SEQ ID NO: 466 - protein, 15 aa, Homo sapiens
SNKEILDEAYVMTNV

SEQ ID NO: 467 - protein, 15 aa, Homo sapiens
SNKEILDEAYVMTSA

SEQ ID NO: 468 - protein, 15 aa, Homo sapiens
SNKEILDEAYVMTSG

SEQ ID NO: 469 - protein, 15 aa, Homo sapiens
SNKEILDEAYVMTSV

SEQ ID NO: 470 - protein, 15 aa, Homo sapiens
SSKDILDEAYVMACA

SEQ ID NO: 471 - protein, 15 aa, Homo sapiens
SSKDILDEAYVMACG

SEQ ID NO: 472 - protein, 15 aa, Homo sapiens
SSKDILDEAYVMACV

SEQ ID NO: 473 - protein, 15 aa, Homo sapiens
SSKDILDEAYVMAIA

SEQ ID NO: 474 - protein, 15 aa, Homo sapiens
SSKDILDEAYVMAIG

SEQ ID NO: 475 - protein, 15 aa, Homo sapiens
SSKDILDEAYVMAIV

SEQ ID NO: 476 - protein, 15 aa, Homo sapiens
SSKDILDEAYVMANA

SEQ ID NO: 477 - protein, 15 aa, Homo sapiens
SSKDILDEAYVMANG

SEQ ID NO: 478 - protein, 15 aa, Homo sapiens
SSKDILDEAYVMANV

SEQ ID NO: 479 - protein, 15 aa, Homo sapiens
SSKDILDEAYVMASA

SEQ ID NO: 480 - protein, 15 aa, Homo sapiens
SSKDILDEAYVMASG

SEQ ID NO: 481 - protein, 15 aa, Homo sapiens
SSKDILDEAYVMASV

SEQ ID NO: 482 - protein, 15 aa, Homo sapiens
SSKDILDEAYVMTCA

SEQ ID NO: 483 - protein, 15 aa, Homo sapiens
SSKDILDEAYVMTCG

SEQ ID NO: 484 - protein, 15 aa, Homo sapiens
SSKDILDEAYVMTCV

SEQ ID NO: 485 - protein, 15 aa, Homo sapiens
SSKDILDEAYVMTIA

SEQ ID NO: 486 - protein, 15 aa, Homo sapiens
SSKDILDEAYVMTIG

SEQ ID NO: 487 - protein, 15 aa, Homo sapiens
SSKDILDEAYVMTIV

SEQ ID NO: 488 - protein, 15 aa, Homo sapiens
SSKDILDEAYVMTNA

SEQ ID NO: 489 - protein, 15 aa, Homo sapiens
SSKDILDEAYVMTNG

SEQ ID NO: 490 - protein, 15 aa, Homo sapiens
SSKDILDEAYVMTNV

SEQ ID NO: 491 - protein, 15 aa, Homo sapiens
SSKDILDEAYVMTSA

SEQ ID NO: 492 - protein, 15 aa, Homo sapiens
SSKDILDEAYVMTSG

SEQ ID NO: 493 - protein, 15 aa, Homo sapiens
SSKDILDEAYVMTSV

SEQ ID NO: 494 - protein, 15 aa, Homo sapiens
SSKEILDEAYVMACA

SEQ ID NO: 495 - protein, 15 aa, Homo sapiens
SSKEILDEAYVMACG

SEQ ID NO: 496 - protein, 15 aa, Homo sapiens
SSKEILDEAYVMACV

SEQ ID NO: 497 - protein, 15 aa, Homo sapiens
SSKEILDEAYVMAIA

SEQ ID NO: 498 - protein, 15 aa, Homo sapiens
SSKEILDEAYVMAIG

SEQ ID NO: 499 - protein, 15 aa, Homo sapiens
SSKEILDEAYVMAIV

SEQ ID NO: 500 - protein, 15 aa, Homo sapiens
SSKEILDEAYVMANA

SEQ ID NO: 501 - protein, 15 aa, Homo sapiens
SSKEILDEAYVMANG

SEQ ID NO: 502 - protein, 15 aa, Homo sapiens
SSKEILDEAYVMANV

SEQ ID NO: 503 - protein, 15 aa, Homo sapiens
SSKEILDEAYVMASA

SEQ ID NO: 504 - protein, 15 aa, Homo sapiens
SSKEILDEAYVMASG

SEQ ID NO: 505 - protein, 15 aa, Homo sapiens
SSKEILDEAYVMASV

SEQ ID NO: 506 - protein, 15 aa, Homo sapiens
SSKEILDEAYVMTCA

SEQ ID NO: 507 - protein, 15 aa, Homo sapiens
SSKEILDEAYVMTCG

SEQ ID NO: 508 - protein, 15 aa, Homo sapiens
SSKEILDEAYVMTCV

SEQ ID NO: 509 - protein, 15 aa, Homo sapiens
SSKEILDEAYVMTIA

SEQ ID NO: 510 - protein, 15 aa, Homo sapiens
SSKEILDEAYVMTIG

SEQ ID NO: 511 - protein, 15 aa, Homo sapiens
SSKEILDEAYVMTIV

SEQ ID NO: 512 - protein, 15 aa, Homo sapiens
SSKEILDEAYVMTNA

SEQ ID NO: 513 - protein, 15 aa, Homo sapiens
SSKEILDEAYVMTNG

SEQ ID NO: 514 - protein, 15 aa, Homo sapiens
SSKEILDEAYVMTNV

SEQ ID NO: 515 - protein, 15 aa, Homo sapiens
SSKEILDEAYVMTSA

SEQ ID NO: 516 - protein, 15 aa, Homo sapiens
SSKEILDEAYVMTSG

SEQ ID NO: 517 - protein, 15 aa, Homo sapiens
SSKEILDEAYVMTSV

SEQ ID NO: 518 - protein, 14 aa, Homo sapiens
AVVMASVDNPHVCR

SEQ ID NO: 519 - protein, 14 aa, Homo sapiens
AYVMASVDNPHVCR

SEQ ID NO: 520 - protein, 13 aa, Homo sapiens
CTGPGLEGCPTNG

SEQ ID NO: 521 - protein, 19 aa, Homo sapiens
DEAYVMASVDNPHVCRLLG

SEQ ID NO: 522 - protein, 20 aa, Homo sapiens
ILKETEFKKIFVLGPGAFGT

SEQ ID NO: 523 - protein, 20 aa, Homo sapiens
ILKETEFKKIFVLGSGAFGT

SEQ ID NO: 524 - protein, 20 aa, Homo sapiens
ILKETEFKKIKVLGPGAFGT

SEQ ID NO: 525 - protein, 20 aa, Homo sapiens
ILKETEFKKIKVLGSGAFGT

SEQ ID NO: 526 - protein, 20 aa, Homo sapiens
ILKETEFKKLFVLGPGAFGT

SEQ ID NO: 527 - protein, 20 aa, Homo sapiens
ILKETEFKKLFVLGSGAFGT

SEQ ID NO: 528 - protein, 20 aa, Homo sapiens
ILKETEFKKLKVLGPGAFGT

SEQ ID NO: 529 - protein, 20 aa, Homo sapiens
ILKETEFKKLKVLGSGAFGT

SEQ ID NO: 530 - protein, 20 aa, Homo sapiens
ILKETELKKIFVLGPGAFGT

SEQ ID NO: 531 - protein, 20 aa, Homo sapiens
ILKETELKKIFVLGSGAFGT

SEQ ID NO: 532 - protein, 20 aa, Homo sapiens
ILKETELKKIKVLGPGAFGT

SEQ ID NO: 533 - protein, 20 aa, Homo sapiens
ILKETELKKIKVLGSGAFGT

SEQ ID NO: 534 - protein, 20 aa, Homo sapiens
ILKETELKKLFVLGPGAFGT

SEQ ID NO: 535 - protein, 20 aa, Homo sapiens
ILKETELKKLFVLGSGAFGT

SEQ ID NO: 536 - protein, 20 aa, Homo sapiens
ILKETELKKLKVLGPGAFGT

SEQ ID NO: 537 - protein, 20 aa, Homo sapiens
ILKETELKKLKVLGSGAFGT

SEQ ID NO: 538 - protein, 20 aa, Homo sapiens
ILMETEFKKIFVLGPGAFGT

SEQ ID NO: 539 - protein, 20 aa, Homo sapiens
ILMETEFKKIFVLGSGAFGT

SEQ ID NO: 540 - protein, 20 aa, Homo sapiens
ILMETEFKKIKVLGPGAFGT

SEQ ID NO: 541 - protein, 20 aa, Homo sapiens
ILMETEFKKIKVLGSGAFGT

SEQ ID NO: 542 - protein, 20 aa, Homo sapiens
ILMETEFKKLFVLGPGAFGT

SEQ ID NO: 543 - protein, 20 aa, Homo sapiens
ILMETEFKKLFVLGSGAFGT

SEQ ID NO: 544 - protein, 20 aa, Homo sapiens
ILMETEFKKLKVLGPGAFGT

SEQ ID NO: 545 - protein, 20 aa, Homo sapiens
ILMETEFKKLKVLGSGAFGT

SEQ ID NO: 546 - protein, 20 aa, Homo sapiens
ILMETELKKIFVLGPGAFGT

SEQ ID NO: 547 - protein, 20 aa, Homo sapiens
ILMETELKKIFVLGSGAFGT

SEQ ID NO: 548 - protein, 20 aa, Homo sapiens
ILMETELKKIKVLGPGAFGT

SEQ ID NO: 549 - protein, 20 aa, Homo sapiens
ILMETELKKIKVLGSGAFGT

SEQ ID NO: 550 - protein, 20 aa, Homo sapiens
ILMETELKKLFVLGPGAFGT

SEQ ID NO: 551 - protein, 20 aa, Homo sapiens
ILMETELKKLFVLGSGAFGT

SEQ ID NO: 552 - protein, 20 aa, Homo sapiens
ILMETELKKLKVLGPGAFGT

SEQ ID NO: 553 - protein, 20 aa, Homo sapiens
ILMETELKKLKVLGSGAFGT

SEQ ID NO: 554 - protein, 15 aa, Homo sapiens
KGNYVVTDHGSCVRA

SEQ ID NO: 555 - protein, 13 aa, Homo sapiens
KVKIPVAIKAPKA

SEQ ID NO: 556 - protein, 19 aa, Homo sapiens
KVKIPVAIKELREATSPKA

SEQ ID NO: 557 - protein, 13 aa, Homo sapiens
KVKIPVAIKSPKA

SEQ ID NO: 558 - protein, 13 aa, Homo sapiens
LLRILKETEFKKI

SEQ ID NO: 559 - protein, 13 aa, Homo sapiens
LLRILKETESKKI

SEQ ID NO: 560 - protein, 13 aa, Homo sapiens
MNYLEDRLLVHRD

SEQ ID NO: 561 - protein, 13 aa, Homo sapiens
MNYLEDRRLVHRD

SEQ ID NO: 562 - protein, 25 aa, Homo sapiens
RLLQERELLEPLTPSGEAPNQAFLR

SEQ ID NO: 563 - protein, 25 aa, Homo sapiens
RLLQERELLEPLTPSGEAPNQALLR

SEQ ID NO: 564 - protein, 25 aa, Homo sapiens
RLLQERELLEPLTPSGEAPNQAPLR

SEQ ID NO: 565 - protein, 25 aa, Homo sapiens
RLLQERELVEPLTPSGEAPNQAFLR

SEQ ID NO: 566 - protein, 25 aa, Homo sapiens
RLLQERELVEPLTPSGEAPNQALLR

SEQ ID NO: 567 - protein, 25 aa, Homo sapiens
RLLQERELVEPLTPSGEAPNQAPLR

SEQ ID NO: 568 - protein, 20 aa, Homo sapiens
TLKETEFKKIFVLGPGAFGT

SEQ ID NO: 569 - protein, 20 aa, Homo sapiens
TLKETEFKKIFVLGSGAFGT

SEQ ID NO: 570 - protein, 20 aa, Homo sapiens
TLKETEFKKIKVLGPGAFGT

SEQ ID NO: 571 - protein, 20 aa, Homo sapiens
TLKETEFKKIKVLGSGAFGT

SEQ ID NO: 572 - protein, 20 aa, Homo sapiens
TLKETEFKKLFVLGPGAFGT

SEQ ID NO: 573 - protein, 20 aa, Homo sapiens
TLKETEFKKLFVLGSGAFGT

SEQ ID NO: 574 - protein, 20 aa, Homo sapiens
TLKETEFKKLKVLGPGAFGT

SEQ ID NO: 575 - protein, 20 aa, Homo sapiens
TLKETEFKKLKVLGSGAFGT

SEQ ID NO: 576 - protein, 20 aa, Homo sapiens
TLKETELKKIFVLGPGAFGT

SEQ ID NO: 577 - protein, 20 aa, Homo sapiens
TLKETELKKIFVLGSGAFGT

SEQ ID NO: 578 - protein, 20 aa, Homo sapiens
TLKETELKKIKVLGPGAFGT

SEQ ID NO: 579 - protein, 20 aa, Homo sapiens
TLKETELKKIKVLGSGAFGT

SEQ ID NO: 580 - protein, 20 aa, Homo sapiens
TLKETELKKLFVLGPGAFGT

SEQ ID NO: 581 - protein, 20 aa, Homo sapiens
TLKETELKKLFVLGSGAFGT

SEQ ID NO: 582 - protein, 20 aa, Homo sapiens
TLKETELKKLKVLGPGAFGT

SEQ ID NO: 583 - protein, 20 aa, Homo sapiens
TLKETELKKLKVLGSGAFGT

SEQ ID NO: 584 - protein, 20 aa, Homo sapiens
TLMETEFKKIFVLGPGAFGT

SEQ ID NO: 585 - protein, 20 aa, Homo sapiens
TLMETEFKKIFVLGSGAFGT

SEQ ID NO: 586 - protein, 20 aa, Homo sapiens
TLMETEFKKIKVLGPGAFGT

SEQ ID NO: 587 - protein, 20 aa, Homo sapiens
TLMETEFKKIKVLGSGAFGT

SEQ ID NO: 588 - protein, 20 aa, Homo sapiens
TLMETEFKKLFVLGPGAFGT

SEQ ID NO: 589 - protein, 20 aa, Homo sapiens
TLMETEFKKLFVLGSGAFGT

SEQ ID NO: 590 - protein, 20 aa, Homo sapiens
TLMETEFKKLKVLGPGAFGT

SEQ ID NO: 591 - protein, 20 aa, Homo sapiens
TLMETEFKKLKVLGSGAFGT

SEQ ID NO: 592 - protein, 20 aa, Homo sapiens
TLMETELKKIFVLGPGAFGT

SEQ ID NO: 593 - protein, 20 aa, Homo sapiens
TLMETELKKIFVLGSGAFGT

SEQ ID NO: 594 - protein, 20 aa, Homo sapiens
TLMETELKKIKVLGPGAFGT

SEQ ID NO: 595 - protein, 20 aa, Homo sapiens
TLMETELKKIKVLGSGAFGT

SEQ ID NO: 596 - protein, 20 aa, Homo sapiens
TLMETELKKLFVLGPGAFGT

SEQ ID NO: 597 - protein, 20 aa, Homo sapiens
TLMETELKKLFVLGSGAFGT

SEQ ID NO: 598 - protein, 20 aa, Homo sapiens
TLMETELKKLKVLGPGAFGT

SEQ ID NO: 599 - protein, 20 aa, Homo sapiens
TLMETELKKLKVLGSGAFGT

SEQ ID NO: 600 - protein, 14 aa, Homo sapiens
VKITDFGLAKLLGA

SEQ ID NO: 601 - protein, 14 aa, Homo sapiens
VKITDFGRAKLLGA

SEQ ID NO: 602 - protein, 13 aa, Homo sapiens
YLEDRRLVHRDLA

SEQ ID NO: 603 - protein, 19 aa, Homo sapiens
KVKIPVAIKELREATSPKA

SEQ ID NO: 604 - protein, 13 aa, Homo sapiens
KVKIPVAIKAPKA

SEQ ID NO: 605 - protein, 13 aa, Homo sapiens
KVKIPVAIKAPTS

SEQ ID NO: 606 - protein, 13 aa, Homo sapiens
KVKIPVAIKDPKA

SEQ ID NO: 607 - protein, 13 aa, Homo sapiens
KVKIPVAIKELKA

SEQ ID NO: 608 - protein, 13 aa, Homo sapiens
KVKIPVAIKEPKA

SEQ ID NO: 609 - protein, 13 aa, Homo sapiens
KVKIPVAIKEQKA

SEQ ID NO: 610 - protein, 13 aa, Homo sapiens
KVKIPVAIKESKA

SEQ ID NO: 611 - protein, 13 aa, Homo sapiens
KVKIPVAIKEVPK

SEQ ID NO: 612 - protein, 13 aa, Homo sapiens
KVKIPVAIKIPKA

SEQ ID NO: 613 - protein, 13 aa, Homo sapiens
KVKIPVAIKSPKA

SEQ ID NO: 614 - protein, 13 aa, Homo sapiens
KVKIPVAIKTPKA

SEQ ID NO: 615 - protein, 13 aa, Homo sapiens
KIPVAIKEASPKA

SEQ ID NO: 616 - protein, 13 aa, Homo sapiens
KIPVAIKEFSPKA

SEQ ID NO: 617 - protein, 13 aa, Homo sapiens
KIPVAIKENSPKA

SEQ ID NO: 618 - protein, 13 aa, Homo sapiens
KIPVAIKVASPKA

SEQ ID NO: 619 - protein, 13 aa, Homo sapiens
KIPVAIKVPSPKA

SEQ ID NO: 620 - protein, 14 aa, Artificial Sequence
  Description of Artificial Sequence Synthetic peptide
VRKSKKSEGPSXKV

SEQ ID NO: 621 - protein, 17 aa, Artificial Sequence
  Description of Artificial Sequence Synthetic peptide
GEKVKIPVAIKSPKANK

SEQ ID NO: 622 - protein, 13 aa, Artificial Sequence
  Description of Artificial Sequence Synthetic peptide
KIPVAIKXPTSPK

SEQ ID NO: 623 - protein, 13 aa, Artificial Sequence
  Description of Artificial Sequence Synthetic peptide
KVKIPVAIKXPKA

SEQ ID NO: 624 - protein, 14 aa, Artificial Sequence
  Description of Artificial Sequence Synthetic peptide
AXVMASVDNPHVCR

SEQ ID NO: 625 - protein, 13 aa, Artificial Sequence
  Description of Artificial Sequence Synthetic peptide
LLRILKETEXKKI

SEQ ID NO: 626 - protein, 13 aa, Artificial Sequence
  Description of Artificial Sequence Synthetic peptide
MNYLEDRXLVHRD

SEQ ID NO: 627 - protein, 45 aa, Artificial Sequence
  Description of Artificial Sequence Synthetic polypeptide
RLLQERELXEPLTPSGEAPNQAXLRXLXETEXKKXXVLGXGAFGT

(19) United States
(12) Patent Application Publication
(10) Pub. No.: US 2013/0331287 A1
(43) Pub. Date: Dec. 12, 2013

(54) Autoimmune Antibodies

(71) Applicant: Hoffmann-La Roche Inc., Nutley, NJ (US)

(72) Inventors: Peter Berndt, Basel (CH); Barbara Klughammer, Rheinfelden (DE)

(21) Appl. No.: 13/911,392

(22) Filed: Jun. 6, 2013

(30) Foreign Application Priority Data
Jun. 7, 2012 (EP) 12171126.1

(51) Int. Cl. G01N 33/68; C07K 16/28

(52) U.S. Cl. CPC G01N 33/6854; C07K 16/2863
USPC 506/9; 436/501; 544/293; 530/389.7; 506/16

(57) ABSTRACT

The invention generally relates to biomarkers associated with NSCLC, and methods and compositions for the detection and diagnosis of non-small cell lung cancer in a human subject.

### RELATED APPLICATIONS

This application claims the benefit of EP Application No. 12171126.1, filed Jun. 7, 2012. All the teachings of the above-referenced application is incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 3, 2013, is named P4928SeqList.txt and is 128,129 bytes in size.

### FIELD OF THE INVENTION

The present invention provides methods for identifying patients diagnosed with non-small cell lung cancer who will most benefit from treatment with erlotinib comprising detecting autoantibodies in blood serum of said patients.

### BACKGROUND OF THE INVENTION

Tarceva® is an orally active, potent, inhibitor of the epidermal growth factor receptor (EGFR) tyrosine kinase (TKI).

Erlotinib hydrochloride is the active ingredient in Tarceva®, which is approved as single agent treatment for patients with advanced non-small cell lung cancer (NSCLC) after treatment with chemotherapy, as maintenance treatment for patients not progressing during chemotherapy (1^{st} line maintenance) or after failure of chemotherapy (2^{nd}/3^{rd} line maintenance). Tarceva® is also approved as first line treatment for patients whose tumor harbors an EGFR activating mutation in the EU.

Expression of unusual proteins is common in cancer and viral infection. The mammalian immune system contains a specialized arm that recognizes proteins induced by cellular transformation (neo-antigens) and effectively eliminates cells that express these neo-antigens against which tolerance has not established during development. This arm of the immune system is thought to be effective against viral infections, spontaneous chromosomal and genomic rearrangements caused by errors of the cell division machinery, or carcinogen-induced transforming mutations. While the initial cytotoxic immune response—which is mediated by the recognition of the unusual proteins displayed on MHCI complexes by CD8⁺ T-cells—is fast and effective, a sustained response against virus infections or aberrant cellular clones requires the co-stimulatory effect of CD4⁺ T-helper cells which are activated after presentation of extracellular peptides (which can stem from cells lysed in the first phase of the cytotoxic immune response) via MHCII complexes of professional antigen presenting cells. Additionally, these cells are able to induce a lasting B-cell response and antibodies to viral or aberrant proteins. The infiltration of a tumor with large numbers of CD8⁺ T-cells has shown to be a better prognostic marker than traditional tumor staging in colorectal cancer (Koizumi, Hojo et al. 2007¹), and it has been associated with favorable prognosis in almost all major solid carcinomas independently of cancer cell type. ¹Koizumi, K., S. Hojo, et al. (2007). “Chemokine receptors in cancer metastasis and cancer cell-derived chemokines in host immune response.” Cancer Science 98(11): 1652-1658

Autoantibodies are well known diagnostic entities in cancer. Many cancer autoantibodies are proteins that are normally expressed in embryonic tissues and therefore represent a “foreign” protein against which immune tolerance is not established. The shedding of these proteins to the bloodstream late in cancer history leads to a humoral immune response. A typical example is the common cancer marker CEA (cancer embryonic antigen). Tumors frequently exhibit faulty protein processing. Prominent examples include proteins that are incorrectly cleaved by cell localization proteases (the presence of autoantibodies against the N-terminal sequence of p53 which is normally cleaved after the protein is targeted to the nucleus is one of the most specific biomarkers of lung cancer) or incorrectly glycosylated by cellular glycosylases (anti-MUC1-antibodies against a form of MUC1 that is incorrectly O-glycosylated is a biomarker for a variety of cancers). Even though p53 mutations are frequently observed in tumors, the specificity of the autoantibodies against this protein is almost always against the N-terminus of the protein—connecting the immunogenicity of p53 more to the ectopic expression of a protein that is normally short lived and located in the nucleus.

The detection of natural EGFR autoantibodies in serum of NSCLC patients has been described by Li et al.². Li et al.³ and Chapman et al.⁴ described the detection of natural autoantibodies to p53, HER2, NY-ESO-1, CAGE, MUC1 and GBU4-5. Further, WO2011073905⁵ relates to tumor markers associated with the progression of a cancer disease from a less progressed stage to a more progressed stage. ²Li Yuan et al, Chinese Journal of Lung Cancer, 13(7), 2010, 727-730³Li Yuan et al, Chinese Journal of Lung Cancer, 12(10), 2009, 1999-6187⁴C J Chapman, A Murray, J E McElveen, U Sahin, U Luxemburger, Ö Türeci, R Wiewrodt, A C Barnes, J F Robertson, “Autoantibodies in lung cancer: possibilities for early detection and subsequent cure”, Thorax 2008; 63(3):228-233⁵WO2011073905

Unusual (mutated or expressed in abnormal quantities or locations) tumor proteins can invoke an antibody response. Tumor-induced autoantibodies have frequently been observed and their utility as diagnostic markers has been investigated (Albert and Darnell 2004⁶). While autoantibodies to tumor proteins can be found that have exquisite specificity, most of them lack the required sensitivity for a diagnostic test. ⁶Albert, M. L. and R. B. Darnell (2004). “Paraneoplastic neurological degenerations: Keys to tumor immunity.” Nat. Rev. Cancer 4(1): 36-44

Few reliable epidemiologic and genetic markers that predict erlotinib hydrochloride response are known in the art. In particular, EGFR mutations in exon 18-21 (intracellular kinase domain of the receptor) were described to be linked with better prognosis as well as with better response to TKI treatment (Paz-Ares, Soulieres et al. 2010⁷). The only predictive marker currently known (EGFR activating mutation) is difficult to diagnose as a biopsy of the tumor is needed. At present only about 50% of NSCLC patients are diagnosed with a biopsy (Reck, Hermes et al. 2011⁸). There is a definite need for simpler techniques to detect predictive markers of TKI efficacy. It is desirable to identify the responders at diagnosis in order to have as many patients as possible benefiting from erlotinib treatment as early as possible, while exploring other treatment options for patients not likely to respond. ⁷Paz-Ares, L., D. Soulieres, et al. (2010). “Clinical outcomes in non-small-cell lung cancer patients with EGFR mutations: pooled analysis.” J Cell Mol Med 14(1-2): 51-69⁸Reck, M., A. Hermes, et al. (2011). “Tissue sampling in lung cancer: A review in light of the MERIT experience.” Lung Cancer 74(1): 1-6

### DETAILED DESCRIPTION OF THE INVENTION

Present invention solves that problem in that it provides a method of diagnosis of cancer in a human subject.

Present invention solves that problem in that it provides a method of diagnosis of non-small cell lung cancer in a human subject.

Present invention solves that problem in that it provides a method of diagnosis of non-small cell lung cancer in a human subject by providing antibodies against mutated EGFR sequences.

A patient identified by a method as described herein is a patient, in particular a NSCLC patient who will respond to the treatment with erlotinib or a pharmaceutically acceptable salt thereof, in particular erlotinib hydrochloride.

We surprisingly found that present autoantibodies possess the required sensitivity for a diagnostic test, especially autoantibodies against mutated EGFR sequences.

EGFR is normally bound to the cell membrane and not shed to the bloodstream. EGFR is a normal adult protein that is found in large quantities in some adult tissues. Immune tolerance is expected to be established against this protein and many of its variants. Almost all of the sequences belong to the cytoplasmic part of the molecule and are invisible to professional antigen presenting cells. The mutations affect only a small part of the EGFR molecule.

Unless defined otherwise, all terms used herein have the same meanings as commonly understood by a skilled artisan to which art this invention belongs.

The term “a level of said autoantibody representative for a human subject of a healthy population” refers to an estimate of the mean level of the autoantibody in serum of a population of patients who do not suffer from NSCLC.

The term “a level of said autoantibody representative for a NSCLC patient” refers to an estimate of the mean level of the autoantibody in serum of a population of patients who suffer from NSCLC.

The term “overall survival (OS)” refers to the length of time from diagnosis of disease during and after treatment the patient survives. As the skilled person will appreciate, a patient's overall survival is improved or enhanced, if the patient belongs to a subgroup of patients that has a statistically significant longer mean survival time as compared to another subgroup of patients.

The term “progression-free survival (PFS)” refers to the length of time from diagnosis of disease during and after treatment during which, according to the assessment of the treating physician or investigator, the patient's disease does not become worse, i.e., does not progress. As the skilled person will appreciate, a patient's progression-free survival is improved or enhanced if the patient belongs to a subgroup of patients that has a longer length of time during which the disease does not progress as compared to the average or mean progression free survival time of a control group of similarly situated patients.

The term “patient” refers to any single mammal for which treatment is desired. In particular, the “patient” is a human subject. More particularly, the patient is a human subject suffering from cancer, in particular NSCLC.

The term “autoantibody” is a type of protein manufactured by the immune system of a patient that is directed against one or more of the patient's own proteins.

The term “amino acid” denotes the group of naturally occurring carboxy a-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

As used herein, “therapy” or “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “gene” as used herein comprises variants of the gene. The term “variant” relates to nucleic acid sequences which are substantially similar to the nucleic acid sequences given by the GenBank accession number. The term “substantially similar” is well understood by a person skilled in the art. In particular, a gene variant may be an allele which shows nucleotide exchanges compared to the nucleic acid sequence of the most prevalent allele in the human population. Preferably, such a substantially similar nucleic acid sequence has a sequence similarity to the most prevalent allele of at least 80%, preferably at least 85%, more preferably at least 90%, most preferably at least 95%. The term “variants” is also meant to relate to splice variants.

The term “mutation” refers to changes in a genomic sequence. These random sequences can be defined as sudden and spontaneous changes in the cell. Mutation can result in several different types of change in sequences; these can either have no effect, alter the product of a gene, or prevent the gene from functioning properly or completely. The term “somatic mutation” refers to a change in the genetic structure that is neither inherited nor passed to offspring.

The phrase “recommending a treatment” as used herein refers to using the information or data generated relating to the level or presence of a biomarker or an autoantibody in a sample of a patient to identify the patient as suitably treated or not suitably treated with a therapy. In some embodiment the therapy may comprise a drug. The information or data may be in any form, written, oral or electronic. In some embodiments, using the information or data generated includes communicating, presenting, reporting, storing, sending, transferring, supplying, transmitting, delivering, dispensing, or combinations thereof. In some embodiments, communicating, presenting, reporting, storing, sending, transferring, supplying, transmitting, delivering, dispensing, or combinations thereof are performed by a computing device, analyzer unit or combination thereof. In some further embodiments, communicating, presenting, reporting, storing, sending, transferring, supplying, transmitting, delivering, dispensing, or combinations thereof are performed by a laboratory or medical professional. In some embodiments, the information or data includes a comparison of the level of said biomarker or autoantibody to a reference level. In some embodiments, the information or data includes an indication that said biomarker or autoantibody is present or absent in the sample. In some embodiments, the information or data includes an indication that the patient is suitably treated or not suitably treated with a therapy comprising said drug. In some embodiments, the therapy is erlotinib.

The phrase “providing a diagnosis” as used herein refers to using the information or data generated relating to the level or presence of a biomarker or an autoantibody in a sample of a patient to diagnose a disease in the patent. The information or data may be in any form, written, oral or electronic. In some embodiments, using the information or data generated includes presenting, reporting, storing, sending, transferring, supplying, transmitting, delivering, dispensing, or combinations thereof. In some embodiments, presenting, reporting, storing, sending, transferring, supplying, transmitting, delivering, dispensing, or combinations thereof are performed by a computing device, analyzer unit or combination thereof. In some embodiments, presenting, reporting, storing, sending, transferring, supplying, transmitting, delivering, dispensing, or combinations thereof are performed by a laboratory or medical professional. In some embodiments, the information or data includes a comparison of the level of said biomarker or autoantibody to a reference level. In some embodiments, the information or data includes an indication that said biomarker or autoantibody is present or absent in the sample. In some embodiments, the information or data includes an indication that the patient is diagnosed with said disease. In some embodiments, said disease is non-small cell lung cancer.

During the TASK study, biopsy material had been collected and the tumor cells have been tested for the presence of the most frequent somatic mutations, i.e. a deletion at exon 19, and a point mutation at exon 21. During the TASK study serum samples had been collected at various time points (pre-dose, day 8, day 22 and progression) from all patients and were assessed for autoantibodies against EGFR.

In these patients, autoantigenic peptide sequences that predict development of rash, or prolonged progression free or overall survival inevitably include a set of sequences that are derived from the EGFR sequence starting at position 737 and extending through 756. These peptides include a number of sequence variants, but inevitably include sequences that have a deletion at position 746-750 or close nearby (Table 2).

This corresponds exactly to the position of the deletion in the exon 19 somatic mutation, known to be predictive for outstanding efficacy from treatment with TKI (Rosell et al. 2009⁹, Mok et al. 2009¹⁰). ⁹Rosell et al., N Engl J Med 2009; 361:958-967¹⁰Mok et al., N Engl J Med 2009; 361:947-957

The presence of a somatic EGFR mutation in exon 19 in tumor tissue leads to protein variant against that the immune system has not developed tolerance. The somatic mutation occurs only in the tumor, and therefore, if it induces an autoantibody which can be detected in the patient's serum it can be used to draw conclusions about the presence of an exon 19 mutation or exon 21 mutation in the NSCLC tissue, which is well known to predict increased progression free survival and superiority of tyrosine kinase monotherapy over chemotherapy (Heigener and Reck 2011¹¹). ¹¹Heigener, D. and M. Reck (2011). “Mutations in the epidermal growth factor receptor gene in non-small cell lung cancer: Impact on treatment beyond gefitinib and erlotinib.” Advances in Therapy 28(2): 126-133

The autoantibody can be detected using a standard blood sample from the patient without the need to obtain tumor cells with a biopsy. This is a large advantage over the current practice, as recovery rates of useful tumor samples even in clinical studies do not exceed 50% (Reck, Hermes et al. 2011¹²). ¹²Reck, M., A. Hermes, et al. (2011). “Tissue sampling in lung cancer: A review in light of the MERIT experience.” Lung Cancer 74(1): 1-6

Anti-EGFR autoantibodies can be detected in blood samples of NSCLC patients at higher concentrations than in healthy controls (FIG. 1). In particular, peptide sequences have been identified that yield consecutive regions of high immunogenicity with large differences between patient and healthy controls sera. Consecutive sequence stretches were identified, where ratios of individual peptide signals in more than 30% of the cancer patients to maximum value in controls was larger than 8 (Table 1).

TABLE 1
Consecutive EGFR sequences with high
autoantigenicity in NLSCL patients

| | | Consensus sequence of |
|---|---|---|
| | Sequence | autoantigenic |
| Protein | Region | peptides in the region |
| EGFR | 323-336 | VRKSKKSEGPSxKV |
| EGFR | 546-564 | PREFVENSECIQCHPECL |
| EGFR | 574-591 | RGPDNCIQCAHYIDGPHCVKTCP |
| EGFR | 735-762 | GEKVKIPVAIK[-S]PKANK |
| | Del 1 | |
| EGFR | 739-758 | KIPVAIK[-HRK]PTSPK |
| | Del 2 | |
| EGFR | 793-806 | MPFGCLLDYVREH |
| EGFR | 867-883 | KEYHAEGGKVPIKWM |
| EGFR | 986-1002 | RMHLPSPTDSNFYRA |
| EGFR | 1081-1095 | SIDDTFLPVPEYIN |
| EGFR | 1148-1166 | NSTFDSPAHWAQKGSHQI |

The above sequences can be used for the early diagnosis of NSCLC.

Regression analysis has identified significant evidence that the presence of antibodies to the peptides influence both, progression free survival (PFS) and overall survival (OS) (FIG. 2). Although the number of samples is small in comparison to the number of peptides, many covariates need to be considered and none of the individual peptides reach sufficient statistical significance, we surprisingly found that when combining overlapping information from various independent statistical approaches, selection of a relevant peptide from the many candidates obtained after univariate analysis is possible. FIG. 3 illustrates the process that led to selection of the candidate sequences listed in Table 2. Sequences listed in Table 2 or any continuous subsequences thereof longer than 8 amino acids are candidate sequences.

TABLE 2
Antigenic sequences influencing PFS and OS of NSCLC patients

| | | Consensus sequence of | |
|---|---|---|---|
| | Sequence | autoantigenic | |
| Protein | Region | peptides in the region | Source |
| EGFR | 737-756 | KVKIPVAIKELREATSPKA | PFS ~ Peptide and |
| EGFR | 737-756 Del | KVKIPVAIK------[SA]PKA | treatment in EGFR - |
| | 742-748 | | mutation positive |
| | | | patients with rash |
| EGFR | 763-776 | A[YV]VMASVDNPHVCR | Consensus of |
| EGFR | 703-717 | LLRILKETE[FS]KKI | statistical |
| EGFR | 825-838 | MNYLEDR[RL]LVHRD | analyses for PFS |
| EGFR | 296-309 | KGNYVVTDHGSCVRA | Consensus of |
| EGFR | 628-641 | CTGPGLEGCPTNG | statistical |
| EGFR | 681-727 | RLLQEREL[VL]EPLTPSGEAPNQA[L | analyses for OS |
| | | PF]LR[IT]L[KM]ETE[FL]KK[IL] | |
| | | [KF]VLG[SP]GAFGT | |
| EGFR | 761-780 | DEAYVMASVDNPHVCRLLG | |
| EGFR | 830-843 | YLEDRRLVHRDLA | |

TABLE 3
Examples for peptide sequences with high
predictive potential for prolonged
progression free survival in patients with
EGFR mutations that develop rash

| KVKIPVAIKELREATSPKA | Annotation |
|---|---|
| KVKIPVAIK------APKA | 737_V003 |
| KVKIPVAIK---APTS | 737_V004 |
| KVKIPVAIKD------PKA | 737_V007 |
| KVKIPVAIKELKA | 737_V015 |
| KVKIPVAIKE------PKA | 737_V019 |
| KVKIPVAIKEQKA | 737_V024 |
| KVKIPVAIKESKA | 737_V029 |
| KVKIPVAIKEV-----PK | 737_V037 |
| KVKIPVAIK------IPKA | 737_V039 |
| KVKIPVAIK------SPKA | 737_V054 |
| KVKIPVAIK------TPKA | 737_V057 |
| --KIPVAIKE----ASPKA | 739 V010 |
| --KIPVAIKEF----SPKA | 739_V013 |
| --KIPVAIKE----NSPKA | 739_V027 |
| --KIPVAIK----VASPKA | 739_V067 |
| --KIPVAIK----VPSPKA | 739_V070 |

EGFR peptide sequences selected from Seq. Id. No. 1-Seq. Id. No. 15 are consecutive sequences with high autoantigenicity in NLSCL patients.

EGFR peptide sequences selected from Seq. Id. No. 16-Seq. Id. No. 517 are useful for predicting the occurrence and grade of adverse events like rash to erlotinib treatment.

EGFR peptide sequences selected from Seq. Id. No. 518-Seq. Id. No. 602 are antigenic sequences influencing, in particular extending, progression free and overall survival of NSCLC patients, in particular mutated EGFR peptide sequences Seq. Id. No. 554, Seq. Id. No. 555, Seq. Id. No. 556, Seq. Id. No. 557, Seq. Id. No. 558, Seq. Id. No. 559 and Seq. Id. No. 560, as well as EGFR peptide sequences Seq. Id. No. 519, Seq. Id. No. 520, Seq. Id. No. 521 and Seq. Id. No. 561.

EGFR peptide sequences selected from Seq. Id. No. 603-Seq. Id. No. 619 have high predictive potential for prolonged progression free survival in patients with EGFR mutations that develop rash.

Antibodies against these peptide sequences most likely influence PFS and OS if they are present in patients. Tests that detect the presence of these antibodies in patient sera could be used to guide treatment and stratify patients into treatment groups.

Present invention provides a method of diagnosis of non-small cell lung cancer in a human subject comprising:

measuring in a blood sample of the human subject a level of an autoantibody selected from the group of autoantibodies recognizing mutated human EGFR, wherein an increased level of said autoantibody selected from the group of autoantibodies recognizing mutated human EGFR in the blood sample of the human subject compared to a level of said autoantibody representative for a human subject of a healthy population is indicative for non-small cell lung cancer.

Present invention provides a method of diagnosis of non-small cell lung cancer in a human subject comprising:

a) measuring in a blood sample of the human subject a level of an autoantibody selected from the group of autoantibodies recognizing mutated human EGFR,

b) comparing the level of said autoantibody to a reference level, and

c) providing a diagnosis of non-small cell lung cancer when the level of said autoantibody is above the reference level.

Present invention provides a method of diagnosis of non-small cell lung cancer in a human subject comprising:

a) measuring in a blood sample of the human subject a level of an autoantibody selected from the group of autoantibodies recognizing mutated human EGFR,

b) comparing the level of said autoantibody to a reference level, and

c) recommending a treatment when the level of said autoantibody is above the reference level.

A certain embodiment of the present invention provides a method as described above, wherein said treatment is erlotinib.

A certain embodiment of present invention provides a method as described herein, wherein the level of an autoantibody recognizing mutated human EGFR is measured.

A certain embodiment of present invention provides a method as described herein, wherein the autoantibody recognizes a mutated EGFR peptide is selected from the group consisting of Seq. Id. No. 1, Seq. Id. No. 2, Seq. Id. No. 4, Seq. Id. No. 5, Seq. Id. No. 6, Seq. Id. No. 7, Seq. Id. No. 15, Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516, Seq. Id. No. 517, Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599, Seq. Id. No. 601, Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

A certain embodiment of present invention provides a method as described herein, wherein the autoantibody recognizes a mutated EGFR peptide that is selected from the groups consisting of Seq. Id. No. 1, Seq. Id. No. 2, Seq. Id. No. 4, Seq. Id. No. 5, Seq. Id. No. 6, Seq. Id. No. 7 and Seq. Id. No. 15.

A certain embodiment of present invention provides a method as described herein, wherein the autoantibody recognizes a mutated EGFR peptide that is selected from the group consisting of Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq.

Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516 and Seq. Id. No. 517.

A certain embodiment of present invention provides a method as described herein, wherein the autoantibody recognizes a mutated EGFR peptide that is selected from the group consisting of Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599 and Seq. Id. No. 601.

A certain embodiment of present invention provides a method as described herein, wherein the autoantibody recognizes a mutated EGFR peptide that is selected from the group consisting of Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

A certain embodiment of present invention provides a method as described herein, wherein the autoantibody recognizes an EGFR peptide that is selected from the group consisting of Seq. Id. No. 519, Seq. Id. No. 520, Seq. Id. No. 521, and Seq. Id. No. 561.

A certain embodiment of present invention provides a method as described herein, wherein the autoantibody recognizes a mutated EGFR peptide that is selected from the group consisting of Seq. Id. No. 554, Seq. Id. No. 555, Seq. Id. No. 556, Seq. Id. No. 557, Seq. Id. No. 558, Seq. Id. No. 559, and Seq. Id. No. 560.

A certain embodiment of present invention provides a method as described herein, wherein the level of an autoantibody in the blood sample of the human subject is 5 times higher than the level of said autoantibody representative for a human subject of a healthy population.

A certain embodiment of present invention provides erlotinib or a pharmaceutically acceptable salt thereof, in particular erlotinib hydrochloride, for use in treating a NSCLC patient identified by a method as described herein comprising administering erlotinib or a pharmaceutically acceptable salt thereof, in particular erlotinib hydrochloride to the patient.

A certain embodiment of present invention provides the use of an autoantibody for predicting the response of a NSCLC patient to erlotinib or a pharmaceutically acceptable salt thereof, in particular erlotinib hydrochloride, treatment, which antibody was identified by a method as described herein.

A certain embodiment of present invention provides a kit for detecting in a blood sample of the human subject a level of one or more autoantibodies selected from the group of autoantibodies recognizing mutated human EGFR, wherein an increased level of said autoantibodies selected from the group of autoantibodies recognizing mutated human EGFR in the blood sample of the human subject compared to a level of said autoantibodies representative for a human subject of a healthy population is indicative for non-small cell lung cancer.

A certain embodiment of present invention provides a kit as described herein, wherein the autoantibody recognizes a mutated EGFR peptide that is selected from the group consisting of Seq. Id. No. 1, Seq. Id. No. 2, Seq. Id. No. 4, Seq. Id. No. 5, Seq. Id. No. 6, Seq. Id. No. 7 and Seq. Id. No. 15.

A certain embodiment of present invention provides a kit as described herein, wherein the autoantibody recognizes a mutated EGFR peptide that is selected from the group consisting of Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516 and Seq. Id. No. 517.

A certain embodiment of present invention provides a kit as described herein, wherein the autoantibody recognizes a mutated EGFR peptide that is selected from the group consisting of Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599 and Seq. Id. No. 601.

A certain embodiment of present invention provides a kit as described herein, wherein the autoantibody recognizes a mutated EGFR peptide that is selected from the group consisting of Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

A certain embodiment of present invention provides a kit as described herein, wherein the autoantibody recognizes a mutated EGFR peptide that is selected from the group consisting of Seq. Id. No. 554, Seq. Id. No. 555, Seq. Id. No. 556, Seq. Id. No. 557, Seq. Id. No. 558, Seq. Id. No. 559, and Seq. Id. No. 560.

A certain embodiment of present invention provides a kit as described herein, wherein the autoantibody recognizes an EGFR peptide that is selected from the group consisting of Seq. Id. No. 519, Seq. Id. No. 520, Seq. Id. No. 521, and Seq. Id. No. 561.

A certain embodiment of present invention provides a method of diagnosis of non-small cell lung cancer in a human subject comprising:

measuring in a blood sample of the human subject a level of an autoantibody selected from the group of autoantibodies recognizing human EGFR, wherein an increased level of said autoantibody selected from the group of autoantibodies recognizing human EGFR in the blood sample of the human subject compared to a level of said autoantibody representative for a human subject of a healthy population is indicative for non-small cell lung cancer, in particular wherein the level of an autoantibody recognizing human EGFR is measured.

Present invention provides a method of diagnosis of non-small cell lung cancer in a human subject comprising:

a) measuring in a blood sample of the human subject a level of an autoantibody selected from the group of autoantibodies recognizing human EGFR,

b) comparing the level of said autoantibody to a reference level, and

c) providing a diagnosis of non-small cell lung cancer when the level of said autoantibody is above the reference level.

Present invention provides a method of diagnosis of non-small cell lung cancer in a human subject comprising:

a) measuring in a blood sample of the human subject a level of an autoantibody selected from the group of autoantibodies recognizing mutated human EGFR,

b) comparing the level of said autoantibody to a reference level, and

c) recommending a treatment when the level of said autoantibody is above the reference level.

A certain embodiment of the present invention provides a method as described above, wherein said treatment is erlotinib.

A certain embodiment of present invention provides a method as described above, wherein the autoantibody recognizes an EGFR peptide is selected from the group consisting of Seq. Id. No. 1, Seq. Id. No. 2, Seq. Id. No. 3, Seq. Id. No. 4, Seq. Id. No. 5, Seq. Id. No. 6, Seq. Id. No. 7, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 15, Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 64, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq.

Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 102, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 309, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 370, Seq. Id. No. 371, Seq. Id. No. 372, Seq. Id. No. 373, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516, Seq. Id. No. 517, Seq. Id. No. 518, Seq. Id. No. 519, Seq. Id. No. 520, Seq. Id. No. 521, Seq. Id. No. 522, Seq. Id. No. 523, Seq. Id. No. 524, Seq. Id. No. 525, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 556, Seq. Id. No. 557, Seq. Id. No. 558, Seq. Id. No. 559, Seq. Id. No. 560, Seq. Id. No. 561, Seq. Id. No. 562, Seq. Id. No. 563, Seq. Id. No. 564, Seq. Id. No. 565, Seq. Id. No. 566, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599, Seq. Id. No. 600, Seq. Id. No. 601, Seq. Id. No. 602, Seq. Id. No. 603, Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618, and Seq. Id. No. 619.

A certain embodiment of present invention provides a method as described above, wherein the autoantibody recognizes an EGFR peptide that is selected from the group consisting of Seq. Id. No. 3, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 64, Seq. Id. No. 102, Seq. Id. No. 309, Seq. Id. No. 370, Seq. Id. No. 373, Seq. Id. No. 519, Seq. Id. No. 520, Seq. Id. No. 521, Seq. Id. No. 525, Seq. Id. No. 556, Seq. Id. No. 558, Seq. Id. No. 561, Seq. Id. No. 566, Seq. Id. No. 600, Seq. Id. No. 602 and Seq. Id. No. 603.

A certain embodiment of present invention provides a kit for detecting in a blood sample of the human subject a level of one or more autoantibodies selected from the group of autoantibodies recognizing human EGFR, wherein an increased level of said autoantibodies selected from the group of autoantibodies recognizing human EGFR in the blood sample of the human subject compared to a level of said autoantibodies representative for a human subject of a healthy population is indicative for non-small cell lung cancer.

A certain embodiment of present invention provides a kit as described above, wherein the autoantibody recognizes an EGFR peptide that is selected from the group consisting of Seq. Id. No. 3, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 64, Seq. Id. No. 102, Seq. Id. No. 309, Seq. Id. No. 370, Seq. Id. No. 373, Seq. Id. No. 519, Seq. Id. No. 520, Seq. Id. No. 521, Seq. Id. No. 525, Seq. Id. No. 556, Seq. Id. No. 558, Seq. Id. No. 561, Seq. Id. No. 566, Seq. Id. No. 600, Seq. Id. No. 602 and Seq. Id. No. 603.

A certain embodiment of present invention provides a kit according as described herein, wherein the autoantibody recognizes a mutated EGFR peptide that is selected from Seq. Id. No. 554, Seq. Id. No. 555, Seq. Id. No. 556, Seq. Id. No. 557, Seq. Id. No. 558, Seq. Id. No. 559, and Seq. Id. No. 560.

A certain embodiment of present invention provides a method as described herein, wherein the autoantibody recognizes an EGFR peptide of Seq. Id. No. 518.

A certain embodiment of present invention provides a method as described herein, wherein the autoantibody recognizes an EGFR peptide of Seq. Id. No. 519.

A certain embodiment of present invention provides a method as described herein, wherein the autoantibody recognizes an EGFR peptide of Seq. Id. No. 520.

A certain embodiment of present invention provides a method as described herein, wherein the autoantibody recognizes an EGFR peptide of Seq. Id. No. 521.

A certain embodiment of present invention provides a method as described herein, wherein the autoantibody recognizes an EGFR peptide of Seq. Id. No. 522.

A certain embodiment of present invention provides a method as described herein, wherein the autoantibody recognizes an EGFR peptide of Seq. Id. No. 523.

A certain embodiment of present invention provides a method as described herein, wherein the autoantibody recognizes an EGFR peptide of Seq. Id. No. 524.

A certain embodiment of present invention provides a method as described herein, wherein the autoantibody recognizes an EGFR peptide of Seq. Id. No. 525.

A certain embodiment of present invention provides a method as described herein, wherein the autoantibody recognizes an EGFR peptide of Seq. Id. No. 526.

A certain embodiment of present invention provides a method as described herein, wherein the autoantibody recognizes an EGFR peptide of Seq. Id. No. 527.

A certain embodiment of present invention provides a method as described herein, wherein the autoantibody recognizes an EGFR peptide of Seq. Id. No. 528.

A certain embodiment of present invention provides a method as described herein, wherein the autoantibody recognizes an EGFR peptide of Seq. Id. No. 529.

A certain embodiment of present invention provides a method as described herein, wherein the autoantibody recognizes an EGFR peptide of Seq. Id. No. 530.

A certain embodiment of present invention provides a method as described herein, wherein the autoantibody recognizes an EGFR peptide of Seq. Id. No. 531.

A certain embodiment of present invention provides a method as described herein, wherein the autoantibody recognizes an EGFR peptide of Seq. Id. No. 532.

A certain embodiment of present invention provides a method as described herein, wherein the autoantibody recognizes an EGFR peptide of Seq. Id. No. 533.

A certain embodiment of present invention provides a method as described herein, wherein the autoantibody recognizes an EGFR peptide of Seq. Id. No. 534.

A certain embodiment of present invention provides a method as described herein, wherein the autoantibody recognizes an EGFR peptide of Seq. Id. No. 535.

A certain embodiment of present invention provides a method as described herein, wherein the autoantibody recognizes an EGFR peptide of Seq. Id. No. 536.

A certain embodiment of present invention provides a method as described herein, wherein the autoantibody recognizes an EGFR peptide of Seq. Id. No. 537.

A certain embodiment of present invention provides a method as described herein, wherein the autoantibody recognizes an EGFR peptide of Seq. Id. No. 538.

A certain embodiment of present invention provides a method as described herein, wherein the autoantibody recognizes an EGFR peptide of Seq. Id. No. 539.

A certain embodiment of present invention provides a method as described herein, wherein the autoantibody recognizes an EGFR peptide of Seq. Id. No. 540.

A certain embodiment of present invention provides a method as described herein, wherein the autoantibody recognizes an EGFR peptide of Seq. Id. No. 541.

A certain embodiment of present invention provides a method as described herein, wherein the autoantibody recognizes an EGFR peptide of Seq. Id. No. 542.

A certain embodiment of present invention provides a method as described herein, wherein the autoantibody recognizes an EGFR peptide of Seq. Id. No. 543.

A certain embodiment of present invention provides a method as described herein, wherein the autoantibody recognizes an EGFR peptide of Seq. Id. No. 544.

A certain embodiment of present invention provides a method as described herein, wherein the autoantibody recognizes an EGFR peptide of Seq. Id. No. 545.

A certain embodiment of present invention provides a method as described herein, wherein the autoantibody recognizes an EGFR peptide of Seq. Id. No. 546.

A certain embodiment of present invention provides a method as described herein, wherein the autoantibody recognizes an EGFR peptide of Seq. Id. No. 547.

A certain embodiment of present invention provides a method as described herein, wherein the autoantibody recognizes an EGFR peptide of Seq. Id. No. 548.

A certain embodiment of present invention provides a method as described herein, wherein the autoantibody recognizes an EGFR peptide of Seq. Id. No. 549.

A certain embodiment of present invention provides a method as described herein, wherein the autoantibody recognizes an EGFR peptide of Seq. Id. No. 550.

A certain embodiment of present invention provides a method as described herein, wherein the autoantibody recognizes an EGFR peptide of Seq. Id. No. 551.

A certain embodiment of present invention provides a method as described herein, wherein the autoantibody recognizes an EGFR peptide of Seq. Id. No. 552.

A certain embodiment of present invention provides a method as described herein, wherein the autoantibody recognizes an EGFR peptide of Seq. Id. No. 553.

A certain embodiment of present invention provides a method as described herein, wherein the autoantibody recognizes an EGFR peptide of Seq. Id. No. 554.

A certain embodiment of present invention provides a method as described herein, wherein the autoantibody recognizes an EGFR peptide of Seq. Id. No. 555.

A certain embodiment of present invention provides a method as described herein, wherein the autoantibody recognizes an EGFR peptide of Seq. Id. No. 556.

A certain embodiment of present invention provides a method as described herein, wherein the autoantibody recognizes an EGFR peptide of Seq. Id. No. 557.

A certain embodiment of present invention provides a method as described herein, wherein the autoantibody recognizes an EGFR peptide of Seq. Id. No. 558.

A certain embodiment of present invention provides a method as described herein, wherein the autoantibody recognizes an EGFR peptide of Seq. Id. No. 559.

A certain embodiment of present invention provides a method as described herein, wherein the autoantibody recognizes an EGFR peptide of Seq. Id. No. 560.

A certain embodiment of present invention provides a method as described herein, wherein the autoantibody recognizes an EGFR peptide of Seq. Id. No. 561.

A certain embodiment of present invention provides a method as described herein, wherein the autoantibody recognizes an EGFR peptide of Seq. Id. No. 562.

A certain embodiment of present invention provides a method as described herein, wherein the autoantibody recognizes an EGFR peptide of Seq. Id. No. 563.

A certain embodiment of present invention provides a method as described herein, wherein the autoantibody recognizes an EGFR peptide of Seq. Id. No. 564.

A certain embodiment of present invention provides a method as described herein, wherein the autoantibody recognizes an EGFR peptide of Seq. Id. No. 565.

A certain embodiment of present invention provides a method as described herein, wherein the autoantibody recognizes an EGFR peptide of Seq. Id. No. 566.

A certain embodiment of present invention provides a method as described herein, wherein the autoantibody recognizes an EGFR peptide of Seq. Id. No. 567.

A certain embodiment of present invention provides a method as described herein, wherein the autoantibody recognizes an EGFR peptide of Seq. Id. No. 568.

A certain embodiment of present invention provides a method as described herein, wherein the autoantibody recognizes an EGFR peptide of Seq. Id. No. 569.

A certain embodiment of present invention provides a method as described herein, wherein the autoantibody recognizes an EGFR peptide of Seq. Id. No. 570.

A certain embodiment of present invention provides a method as described herein, wherein the autoantibody recognizes an EGFR peptide of Seq. Id. No. 571.

A certain embodiment of present invention provides a method as described herein, wherein the autoantibody recognizes an EGFR peptide of Seq. Id. No. 572.

A certain embodiment of present invention provides a method as described herein, wherein the autoantibody recognizes an EGFR peptide of Seq. Id. No. 573.

A certain embodiment of present invention provides a method as described herein, wherein the autoantibody recognizes an EGFR peptide of Seq. Id. No. 574.

A certain embodiment of present invention provides a method as described herein, wherein the autoantibody recognizes an EGFR peptide of Seq. Id. No. 575.

A certain embodiment of present invention provides a method as described herein, wherein the autoantibody recognizes an EGFR peptide of Seq. Id. No. 576.

A certain embodiment of present invention provides a method as described herein, wherein the autoantibody recognizes an EGFR peptide of Seq. Id. No. 577.

A certain embodiment of present invention provides a method as described herein, wherein the autoantibody recognizes an EGFR peptide of Seq. Id. No. 578.

A certain embodiment of present invention provides a method as described herein, wherein the autoantibody recognizes an EGFR peptide of Seq. Id. No. 579.

A certain embodiment of present invention provides a method as described herein, wherein the autoantibody recognizes an EGFR peptide of Seq. Id. No. 580.

A certain embodiment of present invention provides a method as described herein, wherein the autoantibody recognizes an EGFR peptide of Seq. Id. No. 581.

A certain embodiment of present invention provides a method as described herein, wherein the autoantibody recognizes an EGFR peptide of Seq. Id. No. 582.

A certain embodiment of present invention provides a method as described herein, wherein the autoantibody recognizes an EGFR peptide of Seq. Id. No. 583.

A certain embodiment of present invention provides a method as described herein, wherein the autoantibody recognizes an EGFR peptide of Seq. Id. No. 584.

A certain embodiment of present invention provides a method as described herein, wherein the autoantibody recognizes an EGFR peptide of Seq. Id. No. 585.

A certain embodiment of present invention provides a method as described herein, wherein the autoantibody recognizes an EGFR peptide of Seq. Id. No. 586.

A certain embodiment of present invention provides a method as described herein, wherein the autoantibody recognizes an EGFR peptide of Seq. Id. No. 587.

A certain embodiment of present invention provides a method as described herein, wherein the autoantibody recognizes an EGFR peptide of Seq. Id. No. 588.

A certain embodiment of present invention provides a method as described herein, wherein the autoantibody recognizes an EGFR peptide of Seq. Id. No. 589.

A certain embodiment of present invention provides a method as described herein, wherein the autoantibody recognizes an EGFR peptide of Seq. Id. No. 590.

A certain embodiment of present invention provides a method as described herein, wherein the autoantibody recognizes an EGFR peptide of Seq. Id. No. 591.

A certain embodiment of present invention provides a method as described herein, wherein the autoantibody recognizes an EGFR peptide of Seq. Id. No. 592.

A certain embodiment of present invention provides a method as described herein, wherein the autoantibody recognizes an EGFR peptide of Seq. Id. No. 593.

A certain embodiment of present invention provides a method as described herein, wherein the autoantibody recognizes an EGFR peptide of Seq. Id. No. 594.

A certain embodiment of present invention provides a method as described herein, wherein the autoantibody recognizes an EGFR peptide of Seq. Id. No. 595.

A certain embodiment of present invention provides a method as described herein, wherein the autoantibody recognizes an EGFR peptide of Seq. Id. No. 596.

A certain embodiment of present invention provides a method as described herein, wherein the autoantibody recognizes an EGFR peptide of Seq. Id. No. 597.

A certain embodiment of present invention provides a method as described herein, wherein the autoantibody recognizes an EGFR peptide of Seq. Id. No. 598.

A certain embodiment of present invention provides a method as described herein, wherein the autoantibody recognizes an EGFR peptide of Seq. Id. No. 599.

A certain embodiment of present invention provides a method as described herein, wherein the autoantibody recognizes an EGFR peptide of Seq. Id. No. 600.

A certain embodiment of present invention provides a method as described herein, wherein the autoantibody recognizes an EGFR peptide of Seq. Id. No. 601.

A certain embodiment of present invention provides a method as described herein, wherein the autoantibody recognizes an EGFR peptide of Seq. Id. No. 602.

A certain embodiment of present invention provides a method as described herein, wherein the autoantibody recognizes an EGFR peptide of Seq. Id. No. 603.

A certain embodiment of present invention provides a method as described herein, wherein the autoantibody recognizes an EGFR peptide of Seq. Id. No. 604.

A certain embodiment of present invention provides a method as described herein, wherein the autoantibody recognizes an EGFR peptide of Seq. Id. No. 605.

A certain embodiment of present invention provides a method as described herein, wherein the autoantibody recognizes an EGFR peptide of Seq. Id. No. 606.

A certain embodiment of present invention provides a method as described herein, wherein the autoantibody recognizes an EGFR peptide of Seq. Id. No. 607.

A certain embodiment of present invention provides a method as described herein, wherein the autoantibody recognizes an EGFR peptide of Seq. Id. No. 608.

A certain embodiment of present invention provides a method as described herein, wherein the autoantibody recognizes an EGFR peptide of Seq. Id. No. 609.

A certain embodiment of present invention provides a method as described herein, wherein the autoantibody recognizes an EGFR peptide of Seq. Id. No. 610.

A certain embodiment of present invention provides a method as described herein, wherein the autoantibody recognizes an EGFR peptide of Seq. Id. No. 611.

A certain embodiment of present invention provides a method as described herein, wherein the autoantibody recognizes an EGFR peptide of Seq. Id. No. 612.

A certain embodiment of present invention provides a method as described herein, wherein the autoantibody recognizes an EGFR peptide of Seq. Id. No. 613.

A certain embodiment of present invention provides a method as described herein, wherein the autoantibody recognizes an EGFR peptide of Seq. Id. No. 614.

A certain embodiment of present invention provides a method as described herein, wherein the autoantibody recognizes an EGFR peptide of Seq. Id. No. 615.

A certain embodiment of present invention provides a method as described herein, wherein the autoantibody recognizes an EGFR peptide of Seq. Id. No. 616.

A certain embodiment of present invention provides a method as described herein, wherein the autoantibody recognizes an EGFR peptide of Seq. Id. No. 617.

A certain embodiment of present invention provides a method as described herein, wherein the autoantibody recognizes an EGFR peptide of Seq. Id. No. 618.

A certain embodiment of present invention provides a method as described herein, wherein the autoantibody recognizes an EGFR peptide of Seq. Id. No. 619.

A certain embodiment of present invention provides a method as described herein, wherein the level of an autoantibody recognizing p53 is measured.

A certain embodiment of present invention provides a method as described herein, wherein the level of an autoantibody in the blood sample of the human subject is 5 times higher than the level of said autoantibody representative for a human subject of a healthy population.

A certain embodiment of present invention provides a method for determining the EGFR mutation status in a tumor tissue of a human subject suffering from non-small cell lung cancer comprising:

detecting in a blood sample of a human being suffering from non-small cell lung cancer an autoantibody selected from the group of autoantibodies recognizing an human EGFR peptide as described herein, wherein the presence of said autoantibody is indicative for the presence of a mutation of exon 19 in the gene encoding EGFR in human tissue.

A certain embodiment of present invention provides a method for determining the EGFR mutation status in a tumor tissue of a human subject suffering from non-small cell lung cancer comprising:

detecting in a blood sample of a human being suffering from non-small cell lung cancer an autoantibody selected from the group of autoantibodies recognizing an human EGFR peptide as described herein, wherein the presence of said autoantibody is indicative for the presence of a deletion of exon 21 in the gene encoding EGFR in human tissue.

A certain embodiment of present invention provides erlotinib or a pharmaceutically acceptable salt thereof, in particular erlotinib hydrochloride, for use in treating a NSCLC patient identified by a method as described herein comprising administering erlotinib or a pharmaceutically acceptable salt thereof, in particular erlotinib hydrochloride to the patient.

A certain embodiment of present invention provides the use of an autoantibody for predicting the response of a NSCLC patient to erlotinib or a pharmaceutically acceptable salt thereof, in particular erlotinib hydrochloride, treatment, which antibody was identified by a method as described herein.

A certain embodiment of present invention provides a kit for detecting in a blood sample of the human subject a level of one or more autoantibodies selected from the group of autoantibodies recognizing human EGFR, wherein an increased level of said autoantibodies selected from the group of autoantibodies recognizing human EGFR in the blood sample of the human subject compared to a level of said autoantibodies representative for a human subject of a healthy population is indicative for non-small cell lung cancer.

A certain embodiment of present invention provides a kit according as described herein, wherein the autoantibody recognizes an EGFR peptide that is selected from Seq. Id. No. 1-Seq. Id. No. 15.

A certain embodiment of present invention provides a kit according as described herein, wherein the autoantibody recognizes an EGFR peptide that is selected from Seq. Id. No. 16-Seq. Id. No. 517.

A certain embodiment of present invention provides a kit according as described herein, wherein the autoantibody recognizes an EGFR peptide that is selected from Seq. Id. No. 518-Seq. Id. No. 602.

A certain embodiment of present invention provides a kit according as described herein, wherein the autoantibody recognizes an EGFR peptide that is selected from Seq. Id. No. 603-Seq. Id. No. 619.

A certain embodiment of present invention provides a kit according as described herein, wherein the autoantibody recognizes an EGFR peptide that is selected from Seq. Id. No. 519, Seq. Id. No. 520, Seq. Id. No. 521, and Seq. Id. No. 561.

### FIGURES AND SEQ. ID. NUMBERS

FIG. 1: Log-transformed values of peptide binding for all peptides grouped by patient (49 samples, 4-digit numbers) and controls (1-digit numbers). Patients have on average higher signals, with a number of antibodies binding to peptides stronger than any signal in control sera.

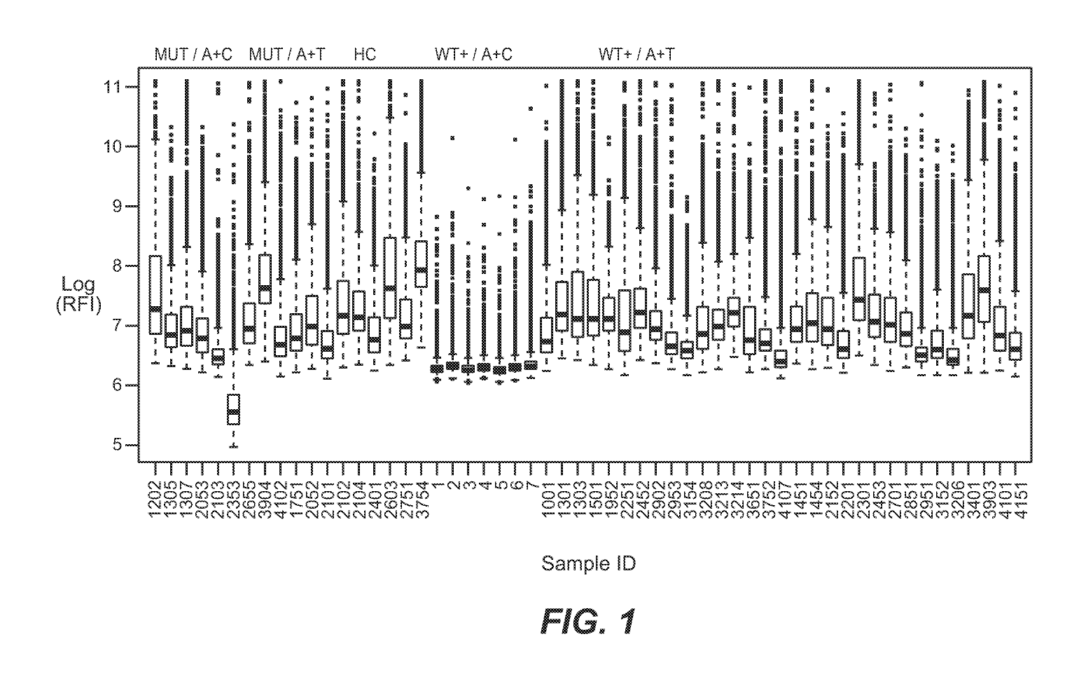

FIG. 2: Histogram of distribution of the p-values for the significance of the antibody titers in a Cox regression model of OS or PFS˜EGFR+TRT+EGFR:TRT+SEX+Antibody titer+ Antibody titer:TRT. If antibody titers do not affect survival, a uniform distribution is expected. The deviation from the uniform distribution is highly significant, which lead to the conclusion that approximately 50% of the 245 antibody titers with p-values<0.05 will have a significant influence on progression free survival. A similar model calculation for overall survival (OS) yields 663 candidates at p-Values<0.05 with a slightly better false discovery rate of approximately 40%.

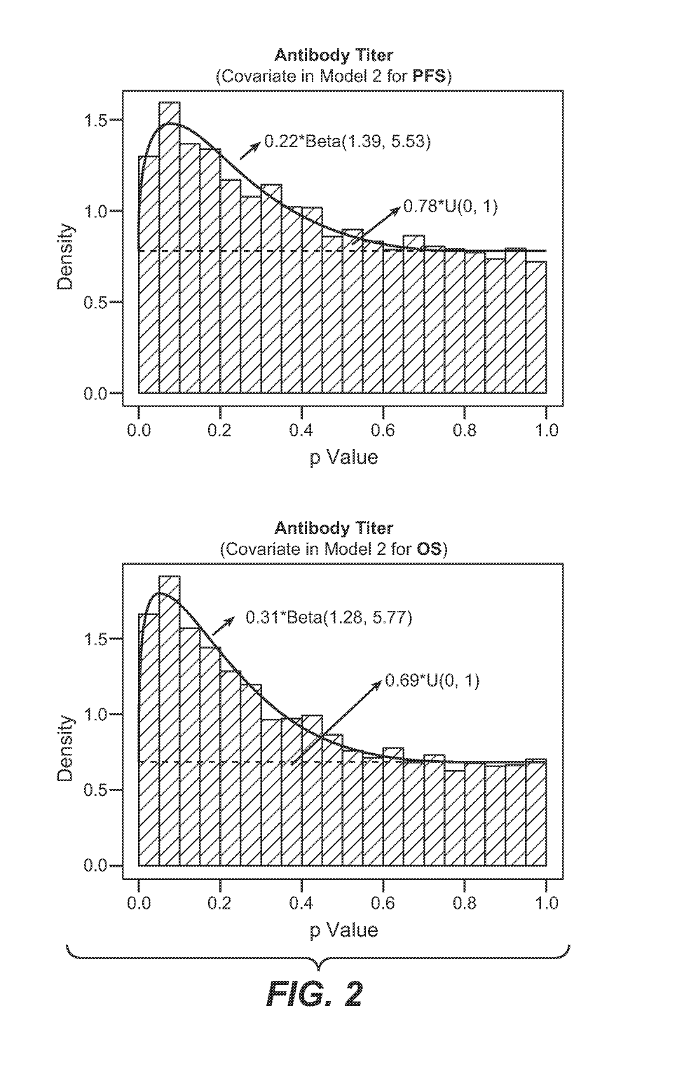

FIG. 3: Venn diagrams illustrating the refinement of peptide candidates for (A) progression free survival and (B) overall survival. Pep—initial peptide selection form Cox-regression model with all covariates; RASH: overlapping sequences from peptides predicting PFS (OS) in patients with rash in a proportional hazard model; TRT: overlapping sequences from peptides predicting PFS (OS) in the erlotinib subgroup; EC4: univariate test of response (after 4 cycles, categorical) vs. antibody titer.

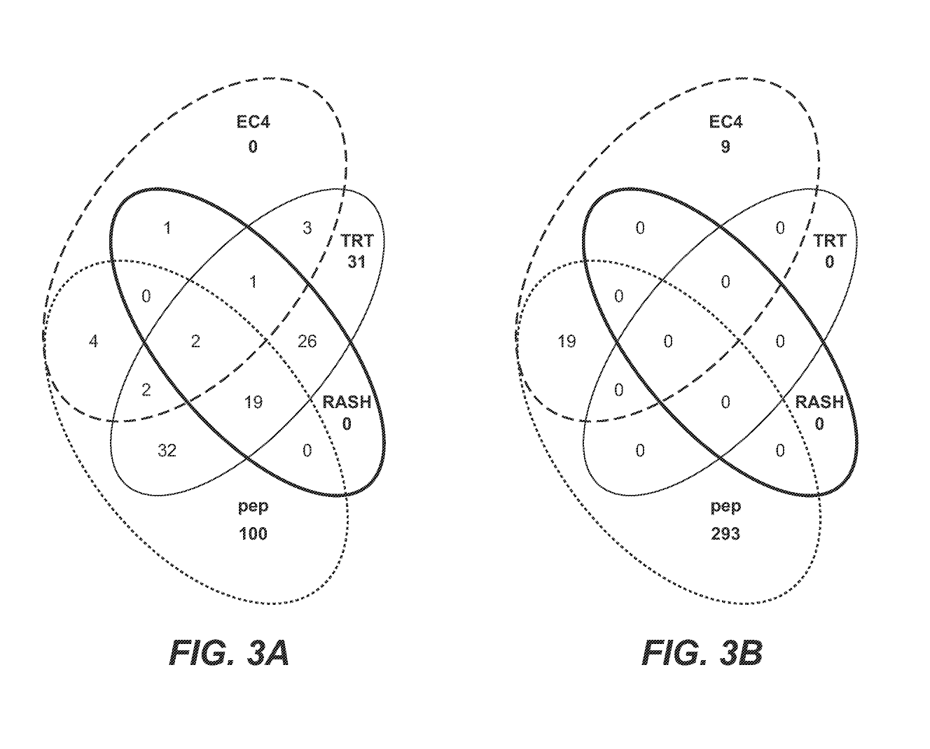

FIG. 4: Presence of autoantibodies against a mutated EGFR peptide predicts (p=0.006) a better treatment outcome in the Tarceva® arm of the trial independent of the occurrence of rash.

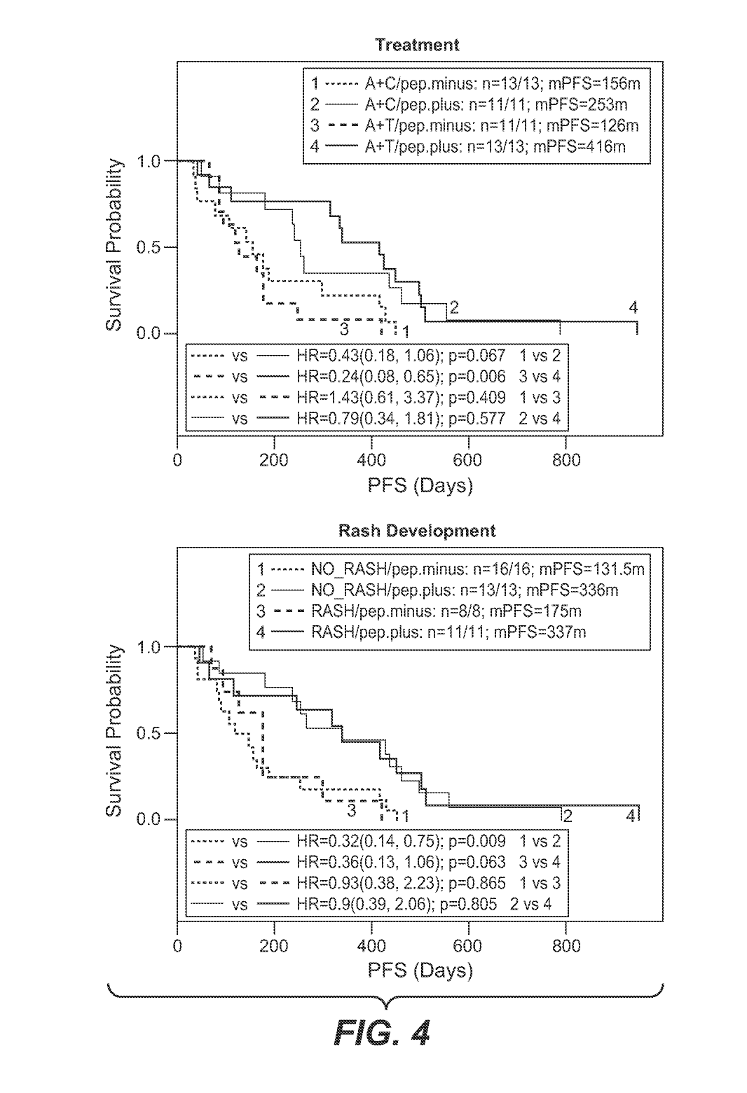

TABLE 4
Seq. Id. No. 1-619 of human EGFR
peptides, *indicates mutated EGFR

| Seq. | |
|---|---|
| Id. No. | protein sequence |
| 1* | GEKVKIPVAIKPKANK |
| 2* | GEKVKIPVAIKSPKANK |
| 3 | KEYHAEGGKVPIKWM |
| 4* | KIPVAIKHPTSPK |
| 5* | KIPVAIKKPTSPK |
| 6* | KIPVAIKPTSPK |
| 7* | KIPVAIKRPTSPK |
| 8 | MPFGCLLDYVREH |
| 9 | NSTFDSPAHWAQKGSHQI |
| 10 | PREFVENSECIQCHPECL |
| 11 | RGPDNCIQCAHYIDGPHCVKTCP |
| 12 | RMHLPSPTDSNFYRA |
| 13 | SIDDTFLPVPEYIN |
| 14 | VRKCKKCEGPCRKV |
| 15* | VRKSKKSEGPSRKV |
| 16* | ADKDILDEAYVMACA |
| 17* | ADKDILDEAYVMACG |
| 18* | ADKDILDEAYVMACV |
| 19* | ADKDILDEAYVMAIA |
| 20* | ADKDILDEAYVMAIG |
| 21* | ADKDILDEAYVMAIV |
| 22* | ADKDILDEAYVMANA |
| 23* | ADKDILDEAYVMANG |
| 24* | ADKDILDEAYVMANV |
| 25* | ADKDILDEAYVMASA |
| 26* | ADKDILDEAYVMASG |
| 27* | ADKDILDEAYVMASV |
| 28* | ADKDILDEAYVMTCA |
| 29* | ADKDILDEAYVMTCG |
| 30* | ADKDILDEAYVMTCV |
| 31* | ADKDILDEAYVMTIA |
| 32* | ADKDILDEAYVMTIG |
| 33* | ADKDILDEAYVMTIV |
| 34* | ADKDILDEAYVMTNA |
| 35* | ADKDILDEAYVMTNG |
| 36* | ADKDILDEAYVMTNV |
| 37* | ADKDILDEAYVMTSA |
| 38* | ADKDILDEAYVMTSG |
| 39* | ADKDILDEAYVMTSV |
| 40* | ADKEILDEAYVMACA |
| 41* | ADKEILDEAYVMACG |
| 42* | ADKEILDEAYVMACV |
| 43* | ADKEILDEAYVMAIA |
| 44* | ADKEILDEAYVMAIG |
| 45* | ADKEILDEAYVMAIV |
| 46* | ADKEILDEAYVMANA |
| 47* | ADKEILDEAYVMANG |
| 48* | ADKEILDEAYVMANV |
| 49* | ADKEILDEAYVMASA |
| 50* | ADKEILDEAYVMASG |
| 51* | ADKEILDEAYVMASV |
| 52* | ADKEILDEAYVMTCA |
| 53* | ADKEILDEAYVMTCG |
| 54* | ADKEILDEAYVMTCV |
| 55* | ADKEILDEAYVMTIA |
| 56* | ADKEILDEAYVMTIG |
| 57* | ADKEILDEAYVMTIV |
| 58* | ADKEILDEAYVMTNA |
| 59* | ADKEILDEAYVMTNG |
| 60* | ADKEILDEAYVMTNV |
| 61* | ADKEILDEAYVMTSA |
| 62* | ADKEILDEAYVMTSG |
| 63* | ADKEILDEAYVMTSV |
| 64 | AIKELREATSPKA |
| 65* | AIKILREATSPKA |
| 66* | AIKVLREATSPKA |
| 67* | ANKDILDEAYVMACA |
| 68* | ANKDILDEAYVMACG |
| 69* | ANKDILDEAYVMACV |
| 70* | ANKDILDEAYVMAIA |
| 71* | ANKDILDEAYVMAIG |
| 72* | ANKDILDEAYVMAIV |
| 73* | ANKDILDEAYVMANA |
| 74* | ANKDILDEAYVMANG |
| 75* | ANKDILDEAYVMANV |
| 76* | ANKDILDEAYVMASA |
| 77* | ANKDILDEAYVMASG |
| 78* | ANKDILDEAYVMASV |
| 79* | ANKDILDEAYVMTCA |
| 80* | ANKDILDEAYVMTCG |
| 81* | ANKDILDEAYVMTCV |
| 82* | ANKDILDEAYVMTIA |
| 83* | ANKDILDEAYVMTIG |
| 84* | ANKDILDEAYVMTIV |
| 85* | ANKDILDEAYVMTNA |
| 86* | ANKDILDEAYVMTNG |
| 87* | ANKDILDEAYVMTNV |
| 88* | ANKDILDEAYVMTSA |
| 89* | ANKDILDEAYVMTSG |
| 90* | ANKDILDEAYVMTSV |
| 91* | ANKEILDEAYVMACA |
| 92* | ANKEILDEAYVMACG |
| 93* | ANKEILDEAYVMACV |
| 94* | ANKEILDEAYVMAIA |
| 95* | ANKEILDEAYVMAIG |
| 96* | ANKEILDEAYVMAIV |
| 97* | ANKEILDEAYVMANA |
| 98* | ANKEILDEAYVMANG |
| 99* | ANKEILDEAYVMANV |
| 100* | ANKEILDEAYVMASA |
| 101* | ANKEILDEAYVMASG |
| 102 | ANKEILDEAYVMASV |
| 103* | ANKEILDEAYVMTCA |
| 104* | ANKEILDEAYVMTCG |
| 105* | ANKEILDEAYVMTCV |
| 106* | ANKEILDEAYVMTIA |
| 107* | ANKEILDEAYVMTIG |
| 108* | ANKEILDEAYVMTIV |
| 109* | ANKEILDEAYVMTNA |
| 110* | ANKEILDEAYVMTNG |
| 111* | ANKEILDEAYVMTNV |
| 112* | ANKEILDEAYVMTSA |
| 113* | ANKEILDEAYVMTSG |
| 114* | ANKEILDEAYVMTSV |
| 115* | ASKDILDEAYVMACA |
| 116* | ASKDILDEAYVMACG |
| 117* | ASKDILDEAYVMACV |
| 118* | ASKDILDEAYVMAIA |
| 119* | ASKDILDEAYVMAIG |
| 120* | ASKDILDEAYVMAIV |
| 121* | ASKDILDEAYVMANA |
| 122* | ASKDILDEAYVMANG |
| 123* | ASKDILDEAYVMANV |
| 124* | ASKDILDEAYVMASA |
| 125* | ASKDILDEAYVMASG |
| 126* | ASKDILDEAYVMASV |
| 127* | ASKDILDEAYVMTCA |
| 128* | ASKDILDEAYVMTCG |
| 129* | ASKDILDEAYVMTCV |
| 130* | ASKDILDEAYVMTIA |
| 131* | ASKDILDEAYVMTIG |
| 132* | ASKDILDEAYVMTIV |
| 133* | ASKDILDEAYVMTNA |
| 134* | ASKDILDEAYVMTNG |
| 135* | ASKDILDEAYVMTNV |
| 136* | ASKDILDEAYVMTSA |
| 137* | ASKDILDEAYVMTSG |
| 138* | ASKDILDEAYVMTSV |
| 139* | ASKEILDEAYVMACA |
| 140* | ASKEILDEAYVMACG |
| 141* | ASKEILDEAYVMACV |
| 142* | ASKEILDEAYVMAIA |
| 143* | ASKEILDEAYVMAIG |
| 144* | ASKEILDEAYVMAIV |
| 145* | ASKEILDEAYVMANA |
| 146* | ASKEILDEAYVMANG |
| 147* | ASKEILDEAYVMANV |
| 148* | ASKEILDEAYVMASA |
| 149* | ASKEILDEAYVMASG |
| 150* | ASKEILDEAYVMASV |
| 151* | ASKEILDEAYVMTCA |
| 152* | ASKEILDEAYVMTCG |
| 153* | ASKEILDEAYVMTCV |
| 154* | ASKEILDEAYVMTIA |
| 155* | ASKEILDEAYVMTIG |
| 156* | ASKEILDEAYVMTIV |
| 157* | ASKEILDEAYVMTNA |
| 158* | ASKEILDEAYVMTNG |
| 159* | ASKEILDEAYVMTNV |
| 160* | ASKEILDEAYVMTSA |
| 161* | ASKEILDEAYVMTSG |
| 162* | ASKEILDEAYVMTSV |
| 163* | CLLVHRDLAARNV |
| 164* | CRLVHRDLAARNV |
| 165* | DDKDILDEAYVMACA |
| 166* | DDKDILDEAYVMACG |
| 167* | DDKDILDEAYVMACV |
| 168* | DDKDILDEAYVMAIA |
| 169* | DDKDILDEAYVMAIG |
| 170* | DDKDILDEAYVMAIV |
| 171* | DDKDILDEAYVMANA |
| 172* | DDKDILDEAYVMANG |
| 173* | DDKDILDEAYVMANV |
| 174* | DDKDILDEAYVMASA |
| 175* | DDKDILDEAYVMASG |
| 176* | DDKDILDEAYVMASV |
| 177* | DDKDILDEAYVMTCA |
| 178* | DDKDILDEAYVMTCG |
| 179* | DDKDILDEAYVMTCV |
| 180* | DDKDILDEAYVMTIA |
| 181* | DDKDILDEAYVMTIG |
| 182* | DDKDILDEAYVMTIV |
| 183* | DDKDILDEAYVMTNA |
| 184* | DDKDILDEAYVMTNG |
| 185* | DDKDILDEAYVMTNV |
| 186* | DDKDILDEAYVMTSA |
| 187* | DDKDILDEAYVMTSG |
| 188* | DDKDILDEAYVMTSV |
| 189* | DDKEILDEAYVMACA |
| 190* | DDKEILDEAYVMACG |
| 191* | DDKEILDEAYVMACV |
| 192* | DDKEILDEAYVMAIA |
| 193* | DDKEILDEAYVMAIG |
| 194* | DDKEILDEAYVMAIV |
| 195* | DDKEILDEAYVMANA |
| 196* | DDKEILDEAYVMANG |
| 197* | DDKEILDEAYVMANV |
| 198* | DDKEILDEAYVMASA |
| 199* | DDKEILDEAYVMASG |
| 200* | DDKEILDEAYVMASV |
| 201* | DDKEILDEAYVMTCA |
| 202* | DDKEILDEAYVMTCG |
| 203* | DDKEILDEAYVMTCV |
| 204* | DDKEILDEAYVMTIA |
| 205* | DDKEILDEAYVMTIG |
| 206* | DDKEILDEAYVMTIV |
| 207* | DDKEILDEAYVMTNA |
| 208* | DDKEILDEAYVMTNG |
| 209* | DDKEILDEAYVMTNV |
| 210* | DDKEILDEAYVMTSA |
| 211* | DDKEILDEAYVMTSG |
| 212* | DDKEILDEAYVMTSV |
| 213* | DNKDILDEAYVMACA |
| 214* | DNKDILDEAYVMACG |
| 215* | DNKDILDEAYVMACV |
| 216* | DNKDILDEAYVMAIA |
| 217* | DNKDILDEAYVMAIG |
| 218* | DNKDILDEAYVMAIV |
| 219* | DNKDILDEAYVMANA |
| 220* | DNKDILDEAYVMANG |
| 221* | DNKDILDEAYVMANV |
| 222* | DNKDILDEAYVMASA |
| 223* | DNKDILDEAYVMASG |
| 224* | DNKDILDEAYVMASV |
| 225* | DNKDILDEAYVMTCA |
| 226* | DNKDILDEAYVMTCG |
| 227* | DNKDILDEAYVMTCV |
| 228* | DNKDILDEAYVMTIA |
| 229* | DNKDILDEAYVMTIG |
| 230* | DNKDILDEAYVMTIV |
| 231* | DNKDILDEAYVMTNA |
| 232* | DNKDILDEAYVMTNG |
| 233* | DNKDILDEAYVMTNV |
| 234* | DNKDILDEAYVMTSA |
| 235* | DNKDILDEAYVMTSG |
| 236* | DNKDILDEAYVMTSV |
| 237* | DNKEILDEAYVMACA |
| 238* | DNKEILDEAYVMACG |
| 239* | DNKEILDEAYVMACV |
| 240* | DNKEILDEAYVMAIA |
| 241* | DNKEILDEAYVMAIG |
| 242* | DNKEILDEAYVMAIV |
| 243* | DNKEILDEAYVMANA |
| 244* | DNKEILDEAYVMANG |
| 245* | DNKEILDEAYVMANV |
| 246* | DNKEILDEAYVMASA |
| 247* | DNKEILDEAYVMASG |
| 248* | DNKEILDEAYVMASV |
| 249* | DNKEILDEAYVMTCA |
| 250* | DNKEILDEAYVMTCG |
| 251* | DNKEILDEAYVMTCV |
| 252* | DNKEILDEAYVMTIA |
| 253* | DNKEILDEAYVMTIG |
| 254* | DNKEILDEAYVMTIV |
| 255* | DNKEILDEAYVMTNA |
| 256* | DNKEILDEAYVMTNG |
| 257* | DNKEILDEAYVMTNV |
| 258* | DNKEILDEAYVMTSA |
| 259* | DNKEILDEAYVMTSG |
| 260* | DNKEILDEAYVMTSV |
| 261* | DSKDILDEAYVMACA |
| 262* | DSKDILDEAYVMACG |
| 263* | DSKDILDEAYVMACV |
| 264* | DSKDILDEAYVMAIA |
| 265* | DSKDILDEAYVMAIG |
| 266* | DSKDILDEAYVMAIV |
| 267* | DSKDILDEAYVMANA |
| 268* | DSKDILDEAYVMANG |
| 269* | DSKDILDEAYVMANV |
| 270* | DSKDILDEAYVMASA |
| 271* | DSKDILDEAYVMASG |
| 272* | DSKDILDEAYVMASV |
| 273* | DSKDILDEAYVMTCA |
| 274* | DSKDILDEAYVMTCG |
| 275* | DSKDILDEAYVMTCV |
| 276* | DSKDILDEAYVMTIA |
| 277* | DSKDILDEAYVMTIG |
| 278* | DSKDILDEAYVMTIV |
| 279* | DSKDILDEAYVMTNA |
| 280* | DSKDILDEAYVMTNG |
| 281* | DSKDILDEAYVMTNV |
| 282* | DSKDILDEAYVMTSA |
| 283* | DSKDILDEAYVMTSG |
| 284* | DSKDILDEAYVMTSV |
| 285* | DSKEILDEAYVMACA |
| 286* | DSKEILDEAYVMACG |
| 287* | DSKEILDEAYVMACV |
| 288* | DSKEILDEAYVMAIA |
| 289* | DSKEILDEAYVMAIG |
| 290* | DSKEILDEAYVMAIV |
| 291* | DSKEILDEAYVMANA |
| 292* | DSKEILDEAYVMANG |
| 293* | DSKEILDEAYVMANV |
| 294* | DSKEILDEAYVMASA |
| 295* | DSKEILDEAYVMASG |
| 296* | DSKEILDEAYVMASV |
| 297* | DSKEILDEAYVMTCA |
| 298* | DSKEILDEAYVMTCG |
| 299* | DSKEILDEAYVMTCV |
| 300* | DSKEILDEAYVMTIA |
| 301* | DSKEILDEAYVMTIG |
| 302* | DSKEILDEAYVMTIV |
| 303* | DSKEILDEAYVMTNA |
| 304* | DSKEILDEAYVMTNG |
| 305* | DSKEILDEAYVMTNV |
| 306* | DSKEILDEAYVMTSA |
| 307* | DSKEILDEAYVMTSG |
| 308* | DSKEILDEAYVMTSV |
| 309 | HYQDPHSTAVGNPEY |
| 310* | KVKIPVAIAPSPKA |
| 311* | KVKIPVAIATSPKA |
| 312* | KVKIPVAIEAPSPKA |
| 313* | KVKIPVAIEATSPKA |
| 314* | KVKIPVAIEEAPSPKA |
| 315* | KVKIPVAIEEATSPKA |
| 316* | KVKIPVAIEEPSPKA |
| 317* | KVKIPVAIEETSPKA |
| 318* | KVKIPVAIEPSPKA |
| 319* | KVKIPVAIERAPSPKA |
| 320* | KVKIPVAIERATSPKA |
| 321* | KVKIPVAIEREAPSPKA |
| 322* | KVKIPVAIEREATSPKA |
| 323* | KVKIPVAIEREPSPKA |
| 324* | KVKIPVAIERETSPKA |
| 325* | KVKIPVAIERPSPKA |
| 326* | KVKIPVAIERTSPKA |
| 327* | KVKIPVAIETSPKA |
| 328* | KVKIPVAIKAPSPKA |
| 329* | KVKIPVAIKATSPKA |
| 330* | KVKIPVAIKEAPSPKA |
| 331* | KVKIPVAIKEATSPKA |
| 332* | KVKIPVAIKEEAPSPKA |
| 333* | KVKIPVAIKEEATSPKA |
| 334* | KVKIPVAIKEEPSPKA |
| 335* | KVKIPVAIKEETSPKA |
| 336* | KVKIPVAIKEPSPKA |
| 337* | KVKIPVAIKERAPSPKA |
| 338* | KVKIPVAIKERATSPKA |
| 339* | KVKIPVAIKEREAPSPKA |
| 340* | KVKIPVAIKEREATSPKA |
| 341* | KVKIPVAIKEREPSPKA |
| 342* | KVKIPVAIKERETSPKA |
| 343* | KVKIPVAIKERPSPKA |
| 344* | KVKIPVAIKERTSPKA |
| 345* | KVKIPVAIKETSPKA |
| 346* | KVKIPVAIKPSPKA |
| 347* | KVKIPVAIKRAPSPKA |
| 348* | KVKIPVAIKRATSPKA |
| 349* | KVKIPVAIKREAPSPKA |
| 350* | KVKIPVAIKREATSPKA |
| 351* | KVKIPVAIKREPSPKA |
| 352* | KVKIPVAIKRETSPKA |
| 353* | KVKIPVAIKRPSPKA |
| 354* | KVKIPVAIKRTSPKA |
| 355* | KVKIPVAIKTSPKA |
| 356* | KVKIPVAIPSPKA |
| 357* | KVKIPVAIRAPSPKA |
| 358* | KVKIPVAIRATSPKA |
| 359* | KVKIPVAIREAPSPKA |
| 360* | KVKIPVAIREATSPKA |
| 361* | KVKIPVAIREP SPKA |
| 362* | KVKIPVAIRETSPKA |
| 363* | KVKIPVAIRPSPKA |
| 364* | KVKIPVAIRTSPKA |
| 365* | KVKIPVAITSPKA |
| 366* | LREEILDEAYVMA |
| 367* | LREPILDEAYVMA |
| 368* | PEGEKAKIPVAIKELREA |
| 369* | PEGEKAKIPVAIRELREA |
| 370 | PEGEKVKIPVAIKELREA |
| 371* | PEGEKVKIPVAIRELREA |
| 372* | RLLVHRDLAARNV |
| 373 | RRLVHRDLAARNV |
| 374* | SDKDILDEAYVMACA |
| 375* | SDKDILDEAYVMACG |
| 376* | SDKDILDEAYVMACV |
| 377* | SDKDILDEAYVMAIA |
| 378* | SDKDILDEAYVMAIG |
| 379* | SDKDILDEAYVMAIV |
| 380* | SDKDILDEAYVMANA |
| 381* | SDKDILDEAYVMANG |
| 382* | SDKDILDEAYVMANV |
| 383* | SDKDILDEAYVMASA |
| 384* | SDKDILDEAYVMASG |
| 385* | SDKDILDEAYVMASV |
| 386* | SDKDILDEAYVMTCA |
| 387* | SDKDILDEAYVMTCG |
| 388* | SDKDILDEAYVMTCV |
| 389* | SDKDILDEAYVMTIA |
| 390* | SDKDILDEAYVMTIG |
| 391* | SDKDILDEAYVMTIV |
| 392* | SDKDILDEAYVMTNA |
| 393* | SDKDILDEAYVMTNG |
| 394* | SDKDILDEAYVMTNV |
| 395* | SDKDILDEAYVMTSA |
| 396* | SDKDILDEAYVMTSG |
| 397* | SDKDILDEAYVMTSV |
| 398* | SDKEILDEAYVMACA |
| 399* | SDKEILDEAYVMACG |
| 400* | SDKEILDEAYVMACV |
| 401* | SDKEILDEAYVMAIA |
| 402* | SDKEILDEAYVMAIG |
| 403* | SDKEILDEAYVMAIV |
| 404* | SDKEILDEAYVMANA |
| 405* | SDKEILDEAYVMANG |
| 406* | SDKEILDEAYVMANV |
| 407* | SDKEILDEAYVMASA |
| 408* | SDKEILDEAYVMASG |
| 409* | SDKEILDEAYVMASV |
| 410* | SDKEILDEAYVMTCA |
| 411* | SDKEILDEAYVMTCG |
| 412* | SDKEILDEAYVMTCV |
| 413* | SDKEILDEAYVMTIA |
| 414* | SDKEILDEAYVMTIG |
| 415* | SDKEILDEAYVMTIV |
| 416* | SDKEILDEAYVMTNA |
| 417* | SDKEILDEAYVMTNG |
| 418* | SDKEILDEAYVMTNV |
| 419* | SDKEILDEAYVMTSA |
| 420* | SDKEILDEAYVMTSG |
| 421* | SDKEILDEAYVMTSV |
| 422* | SNKDILDEAYVMACA |
| 423* | SNKDILDEAYVMACG |
| 424* | SNKDILDEAYVMACV |
| 425* | SNKDILDEAYVMAIA |
| 426* | SNKDILDEAYVMAIG |
| 427* | SNKDILDEAYVMAIV |
| 428* | SNKDILDEAYVMANA |
| 429* | SNKDILDEAYVMANG |
| 430* | SNKDILDEAYVMANV |
| 431* | SNKDILDEAYVMASA |
| 432* | SNKDILDEAYVMASG |
| 433* | SNKDILDEAYVMASV |
| 434* | SNKDILDEAYVMTCA |
| 435* | SNKDILDEAYVMTCG |
| 436* | SNKDILDEAYVMTCV |
| 437* | SNKDILDEAYVMTIA |
| 438* | SNKDILDEAYVMTIG |
| 439* | SNKDILDEAYVMTIV |
| 440* | SNKDILDEAYVMTNA |
| 441* | SNKDILDEAYVMTNG |
| 442* | SNKDILDEAYVMTNV |
| 443* | SNKDILDEAYVMTSA |
| 444* | SNKDILDEAYVMTSG |
| 445* | SNKDILDEAYVMTSV |
| 446* | SNKEILDEAYVMACA |
| 447* | SNKEILDEAYVMACG |
| 448* | SNKEILDEAYVMACV |
| 449* | SNKEILDEAYVMAIA |
| 450* | SNKEILDEAYVMAIG |
| 451* | SNKEILDEAYVMAIV |
| 452* | SNKEILDEAYVMANA |
| 453* | SNKEILDEAYVMANG |
| 454* | SNKEILDEAYVMANV |
| 455* | SNKEILDEAYVMASA |
| 456* | SNKEILDEAYVMASG |
| 457* | SNKEILDEAYVMASV |
| 458* | SNKEILDEAYVMTCA |
| 459* | SNKEILDEAYVMTCG |
| 460* | SNKEILDEAYVMTCV |
| 461* | SNKEILDEAYVMTIA |
| 462* | SNKEILDEAYVMTIG |
| 463* | SNKEILDEAYVMTIV |
| 464* | SNKEILDEAYVMTNA |
| 465* | SNKEILDEAYVMTNG |
| 466* | SNKEILDEAYVMTNV |
| 467* | SNKEILDEAYVMTSA |
| 468* | SNKEILDEAYVMTSG |
| 469* | SNKEILDEAYVMTSV |
| 470* | SSKDILDEAYVMACA |
| 471* | SSKDILDEAYVMACG |
| 472* | SSKDILDEAYVMACV |
| 473* | SSKDILDEAYVMAIA |
| 474* | SSKDILDEAYVMAIG |
| 475* | SSKDILDEAYVMAIV |
| 476* | SSKDILDEAYVMANA |
| 477* | SSKDILDEAYVMANG |
| 478* | SSKDILDEAYVMANV |
| 479* | SSKDILDEAYVMASA |
| 480* | SSKDILDEAYVMASG |
| 481* | SSKDILDEAYVMASV |
| 482* | SSKDILDEAYVMTCA |
| 483* | SSKDILDEAYVMTCG |
| 484* | SSKDILDEAYVMTCV |
| 485* | SSKDILDEAYVMTIA |
| 486* | SSKDILDEAYVMTIG |
| 487* | SSKDILDEAYVMTIV |
| 488* | SSKDILDEAYVMTNA |
| 489* | SSKDILDEAYVMTNG |
| 490* | SSKDILDEAYVMTNV |
| 491* | SSKDILDEAYVMTSA |
| 492* | SSKDILDEAYVMTSG |
| 493* | SSKDILDEAYVMTSV |
| 494* | SSKEILDEAYVMACA |
| 495* | SSKEILDEAYVMACG |
| 496* | SSKEILDEAYVMACV |
| 497* | SSKEILDEAYVMAIA |
| 498* | SSKEILDEAYVMAIG |
| 499* | SSKEILDEAYVMAIV |
| 500* | SSKEILDEAYVMANA |
| 501* | SSKEILDEAYVMANG |
| 502* | SSKEILDEAYVMANV |
| 503* | SSKEILDEAYVMASA |
| 504* | SSKEILDEAYVMASG |
| 505* | SSKEILDEAYVMASV |
| 506* | SKEILDEAYVMTCA |
| 507* | SSKEILDEAYVMTCG |
| 508* | SSKEILDEAYVMTCV |
| 509* | SSKEILDEAYVMTIA |
| 510* | SSKEILDEAYVMTIG |
| 511* | SSKEILDEAYVMTIV |
| 512* | SSKEILDEAYVMTNA |
| 513* | SSKEILDEAYVMTNG |
| 514* | SSKEILDEAYVMTNV |
| 515* | SSKEILDEAYVMTSA |
| 516* | SSKEILDEAYVMTSG |
| 517* | SSKEILDEAYVMTSV |
| 518* | AVVMASVDNPHVCR |
| 519 | AYVMASVDNPHVCR |
| 520 | CTGPGLEGCPTNG |
| 521 | DEAYVMASVDNPHVCRLLG |
| 522* | ILKETEFKKIFVLGPGAFGT |
| 523* | ILKETEFKKIFVLGSGAFGT |
| 524* | ILKETEFKKIKVLGPGAFGT |
| 525 | ILKETEFKKIKVLGSGAFGT |
| 526* | ILKETEFKKLFVLGPGAFGT |
| 527* | ILKETEFKKLFVLGSGAFGT |
| 528* | ILKETEFKKLKVLGPGAFGT |
| 529* | ILKETEFKKLKVLGSGAFGT |
| 530* | ILKETELKKIFVLGPGAFGT |
| 531* | ILKETELKKIFVLGSGAFGT |
| 532* | ILKETELKKIKVLGPGAFGT |
| 533* | ILKETELKKIKVLGSGAFGT |
| 534* | ILKETELKKLFVLGPGAFGT |
| 535* | ILKETELKKLFVLGSGAFGT |
| 536* | ILKETELKKLKVLGPGAFGT |
| 537* | ILKETELKKLKVLGSGAFGT |
| 538* | ILMETEFKKIFVLGPGAFGT |
| 539* | ILMETEFKKIFVLGSGAFGT |
| 540* | ILMETEFKKIKVLGPGAFGT |
| 541* | ILMETEFKKIKVLGSGAFGT |
| 542* | ILMETEFKKLFVLGPGAFGT |
| 543* | ILMETEFKKLFVLGSGAFGT |
| 544* | ILMETEFKKLKVLGPGAFGT |
| 545* | ILMETEFKKLKVLGSGAFGT |
| 546* | ILMETELKKIFVLGPGAFGT |
| 547* | ILMETELKKIFVLGSGAFGT |
| 548* | ILMETELKKIKVLGPGAFGT |
| 549* | ILMETELKKIKVLGSGAFGT |
| 550* | ILMETELKKLFVLGPGAFGT |
| 551* | ILMETELKKLFVLGSGAFGT |
| 552* | ILMETELKKLKVLGPGAFGT |
| 553* | ILMETELKKLKVLGSGAFGT |
| 554* | KGNYVVTDHGSCVRA |
| 555* | KVKIPVAIKAPKA |
| 556 | KVKIPVAIKELREATSPKA |
| 557* | KVKIPVAIKSPKA |
| 558 | LLRILKETEFKKI |
| 559* | LLRILKETESKKI |
| 560* | MNYLEDRLLVHRD |
| 561 | MNYLEDRRLVHRD |
| 562* | RLLQERELLEPLTPSGEAPNQAFLR |
| 563* | RLLQERELLEPLTPSGEAPNQALLR |
| 564* | RLLQERELLEPLTPSGEAPNQAPLR |
| 565* | RLLQERELVEPLTPSGEAPNQAFLR |
| 566 | RLLQERELVEPLTPSGEAPNQALLR |
| 567* | RLLQERELVEPLTPSGEAPNQAPLR |
| 568* | TLKETEFKKIFVLGPGAFGT |
| 569* | TLKETEFKKIFVLGSGAFGT |
| 570* | TLKETEFKKIKVLGPGAFGT |
| 571* | TLKETEFKKIKVLGSGAFGT |
| 572* | TLKETEFKKLFVLGPGAFGT |
| 573* | TLKETEFKKLFVLGSGAFGT |
| 574* | TLKETEFKKLKVLGPGAFGT |
| 575* | TLKETEFKKLKVLGSGAFGT |
| 576* | TLKETELKKIFVLGPGAFGT |
| 577* | TLKETELKKIFVLGSGAFGT |
| 578* | TLKETELKKIKVLGPGAFGT |
| 579* | TLKETELKKIKVLGSGAFGT |
| 580* | TLKETELKKLFVLGPGAFGT |
| 581* | TLKETELKKLFVLGSGAFGT |
| 582* | TLKETELKKLKVLGPGAFGT |
| 583* | TLKETELKKLKVLGSGAFGT |
| 584* | TLMETEFKKIFVLGPGAFGT |
| 585* | TLMETEFKKIFVLGSGAFGT |
| 586* | TLMETEFKKIKVLGPGAFGT |
| 587* | TLMETEFKKIKVLGSGAFGT |
| 588* | TLMETEFKKLFVLGPGAFGT |
| 589* | TLMETEFKKLFVLGSGAFGT |
| 590* | TLMETEFKKLKVLGPGAFGT |
| 591* | TLMETEFKKLKVLGSGAFGT |
| 592* | TLMETELKKIFVLGPGAFGT |
| 593* | TLMETELKKIFVLGSGAFGT |
| 594* | TLMETELKKIKVLGPGAFGT |
| 595* | TLMETELKKIKVLGSGAFGT |
| 596* | TLMETELKKLFVLGPGAFGT |
| 597* | TLMETELKKLFVLGSGAFGT |
| 598* | TLMETELKKLKVLGPGAFGT |
| 599* | TLMETELKKLKVLGSGAFGT |
| 600 | VKITDFGLAKLLGA |
| 601* | VKITDFGRAKLLGA |
| 602 | YLEDRRLVHRDLA |
| 603 | KVKIPVAIKELREATSPKA |
| 604* | KVKIPVAIKAPKA |
| 605* | KVKIPVAIKAPTS |
| 606* | KVKIPVAIKDPKA |
| 607* | KVKIPVAIKELKA |
| 608* | KVKIPVAIKEPKA |
| 609* | KVKIPVAIKEQKA |
| 610* | KVKIPVAIKESKA |
| 611* | KVKIPVAIKEVPK |
| 612* | KVKIPVAIKIPKA |
| 613* | KVKIPVAIKSPKA |
| 614* | KVKIPVAIKTPKA |
| 615* | KIPVAIKEASPKA |
| 616* | KIPVAIKEFSPKA |
| 617* | KIPVAIKENSPKA |
| 618* | KIPVAIKVASPKA |
| 619* | KIPVAIKVPSPKA |

### Experiments

### Peptide Arrays

Peptide arrays were created by PepStar™ (JPT Peptide Technologies GmbH, Berlin, Germany) peptide microarray platform to generate customized peptide microarrays on glass slides for biomarker discovery, immunomonitoring and detection and validation of protein interactions. Peptides are immobilized on glass slides via a flexible linker. Chemoselective coupling generates microarrays of directed and covalently attached peptides.

3661 specified native peptides with 500 scrambled control sequences covering the sequences of EGF-receptor, Arachidonate 15-lipoxygenase B (LX15B) and p53 and variants thereof were synthesized and arrays printed. A serum dilution 1:200 was used for the detection of EGFR binding in NSCLC patient samples. Incubation of microarrays was performed in an automated Hybridization Station HS4800 (Tecan) at 30° C. After washing, bound immunoglobine G (IgG) was detected with Cy5—labeled-anti-human secondary antibody (JIR, 0.1 μg/ml end-conc. in assay). Fluorescence was read using a Microarray Scanner GenePix with Autoloader 4200AL (Molecular Devices). Signal intensity is displayed as relative fluorescence units.

The following tables detail the wash and incubation conditions:

TABLE 5
experimental conditions

| | |
|---|---|
| Blocking Solution: | SmartBlock Blocking Buffer |
| Diluent for Serum and 2nd AB: | SuperBlock T20 Blocking Buffer |
| Wash Buffer 1: | 1x TBS + 0.1% Tween 20 |
| Wash Buffer 2: | 0.1x SSC + 0.05% Tween 20 |

TABLE 6
experimental conditions

| Step | Procedure | Parameters | Buffer |
|---|---|---|---|
| 1 | Wash | 1x | 1x TBS-0, 1% Tween20 |
| 2 | Probe injection 1 | | Blocking Solution |
| 3 | Hybridization | 1 h, 30° C. | |
| 4 | Wash | 2x | 1x TBS-0, 1% Tween20 |
| 5 | Probe injection 2 | 1:200 | Patient Serum |
| 6 | Hybridization | 2 h, 30° C. | |
| 7 | Wash | 3x | 1x TBS-0, 1% Tween20 |
| 8 | Probe injection 3 | | Cy5-anti-human Secondary Antibody |
| 9 | Hybridization | 45 min., | |
| | | 30° C. | |
| 10 | Wash | 2x | 1x TBS-0, 1% Tween20 |
| 11 | Wash | 3x | 0.1x SSC-0.05% Tween20 |
| 12 | Slide drying | 20 sec | |
| 13 | Wash | 3x | 0.1x SSC-0.05% Tween20 |
| 14 | Slide drying | 5 min. | |

### Samples

Samples were selected from patients belonging to the TASK study. TASK was a 200-patient randomized, open label, Phase II study of Tarceva® in combination with Avastin® (bevazizumab) compared to standard chemotherapy regimens (gemcitabine plus cisplatin or paclitaxel plus carboplatin) plus Avastin® in first-line NSCLC patients. Further enrollment on this study was halt after data from a pre-planned interim analysis of the first 120 patients. Occurrence of rash was recorded as adverse event. Biopsies from all patients were tested for the occurrence of EGFR mutations and the mutation status has been recorded.

For the peptide array study, a total of 49 patients from both arms (24 Avastin® and Tarceva® (A+T), 25 Avastin® and chemotherapy (A+C)) have been used. In the patients chosen for the A+T arm, rash occurred in 16 patients. In comparison, only 3 patients in the A+C arm developed rash. This corresponds to the expected frequency of rash, as rash is induced by erlotinib, but only to a very limited extend by chemotherapy.

Clinical data accessible included outcome data, e.g. response overall survival and progression free survival as well as the rash grade and the mutation status of EGFR as measured from a pre-treatment biopsy.

### Statistical Analysis

### Global Test

The following approach to model the data was applied:
Descriptive statistics (using appropriate single variable) tests to identify covariates as candidates for a linear regression model describing the performance parameter (PFS or OS).
Identification of the best model for efficacy parameters (survival analysis)
Addition of antibody titer and its interaction with treatment to the model and comparison of the performance with the new variables
   With a general model selected, tests on antibody titers within groups of covariates of interest were conducted:
Proportional hazard model by EGFR mutation (EGFRM), Thoracic Radiotherapy (TRT), rash of efficacy parameters vs. antibody abundance (two sample groups—low/high abundance—divided by median)
U-tests within wild type (WT+) in A+T arm of antibody titer vs. responders/non-responders
χ2 tests by EGFRM and TRT of responses (weeks 6, 12) vs. antibody abundance

Finally, various very similar peptide sequences that yield highly correlated antibody titers were used. For most of the reported peptide sequences, consensus sequence from a listing of all peptides with very high sequence similarity (Smith-Waterman alignment similarity score >50, using a PAM30 substitution matrix and a gap penalty of 1) was selected and antibody titers were correlated to the target titer (Spearman Rank correlation >0.75).

1. A method of diagnosis of non-small cell lung cancer in a human subject comprising:
measuring in a blood sample of the human subject a level of an autoantibody selected from the group of autoantibodies recognizing mutated human EGFR, wherein an increased level of said autoantibody selected from the group of autoantibodies recognizing mutated human EGFR in the blood sample of the human subject compared to a level of said autoantibody representative for a human subject of a healthy population is indicative for non-small cell lung cancer.

2. The method according to claim 1, wherein the autoantibody recognizes a mutated EGFR peptide is selected from the group consisting of Seq. Id. No. 554, Seq. Id. No. 555, Seq. Id. No. 556, Seq. Id. No. 557, Seq. Id. No. 558, Seq. Id. No. 559, and Seq. Id. No. 560.

3. The method according to any of claims 1-2, wherein the autoantibody recognizes a mutated EGFR peptide is selected from the group consisting of Seq. Id. No. 1, Seq. Id. No. 2, Seq. Id. No. 4, Seq. Id. No. 5, Seq. Id. No. 6, Seq. Id. No. 7, Seq. Id. No. 15, Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516, Seq. Id. No. 517, Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599, Seq. Id. No. 601, Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

4. The method according to any of claims 1-3, wherein the autoantibody recognizes a mutated EGFR peptide that is selected from the groups consisting of Seq. Id. No. 1, Seq. Id. No. 2, Seq. Id. No. 4, Seq. Id. No. 5, Seq. Id. No. 6, Seq. Id. No. 7 and Seq. Id. No. 15.

5. The method according to any of claims 1-3, wherein the autoantibody recognizes a mutated EGFR peptide that is selected from the group consisting of Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516 and Seq. Id. No. 517.

6. The method according to any of claims 1-3, wherein the autoantibody recognizes a mutated EGFR peptide that is selected from the group consisting of Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599 and Seq. Id. No. 601.

7. The method according to any of claims 1-3, wherein the autoantibody recognizes a mutated EGFR peptide that is selected from the group consisting of Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

8. The method according to any of claims 1-7, wherein the level of an autoantibody in the blood sample of the human subject is 5 times higher than the level of said autoantibody representative for a human subject of a healthy population.

9. Erlotinib or a pharmaceutically acceptable salt thereof, in particular erlotinib hydrochloride, for use in treating a NSCLC patient identified by a method of any of claims 1 to 8 comprising administering erlotinib or a pharmaceutically acceptable salt thereof, in particular erlotinib hydrochloride to the patient.

10. Use of an autoantibody for predicting the response of a NSCLC patient to erlotinib or a pharmaceutically acceptable salt thereof, in particular erlotinib hydrochloride, treatment, which antibody was identified by a method of any of claims 1 to 8.

11. A kit for detecting in a blood sample of the human subject a level of one or more autoantibodies selected from the group of autoantibodies recognizing mutated human EGFR, wherein an increased level of said autoantibodies selected from the group of autoantibodies recognizing mutated human EGFR in the blood sample of the human subject compared to a level of said autoantibodies representative for a human subject of a healthy population is indicative for non-small cell lung cancer.

12. A kit according to claim 11, wherein the autoantibody recognizes a mutated EGFR peptide that is selected from the group consisting of Seq. Id. No. 554, Seq. Id. No. 555, Seq. Id. No. 556, Seq. Id. No. 557, Seq. Id. No. 558, Seq. Id. No. 559, and Seq. Id. No. 560.

13. A kit according to claim 11, wherein the autoantibody recognizes a mutated EGFR peptide that is selected from the group consisting of Seq. Id. No. 1, Seq. Id. No. 2, Seq. Id. No. 4, Seq. Id. No. 5, Seq. Id. No. 6, Seq. Id. No. 7 and Seq. Id. No. 15.

14. A kit according to claim 11, wherein the autoantibody recognizes a mutated EGFR peptide that is selected from the group consisting of Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516 and Seq. Id. No. 517.

15. A kit according to claim 11, wherein the autoantibody recognizes a mutated EGFR peptide that is selected from the group consisting of Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599 and Seq. Id. No. 601.

16. A kit according to claim 11, wherein the autoantibody recognizes a mutated EGFR peptide that is selected from the group consisting of Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

17. A method of diagnosis of non-small cell lung cancer in a human subject comprising:
measuring in a blood sample of the human subject a level of an autoantibody selected from the group of autoantibodies recognizing human EGFR, wherein an increased level of said autoantibody selected from the group of autoantibodies recognizing human EGFR in the blood sample of the human subject compared to a level of said autoantibody representative for a human subject of a healthy population is indicative for non-small cell lung cancer.

18. The method according to claim 16, wherein the autoantibody recognizes a mutated EGFR peptide is selected from the group consisting of Seq. Id. No. 519, Seq. Id. No. 520, Seq. Id. No. 521 and Seq. Id. No. 561.

19. The method according to any of claims 17-18, wherein the autoantibody recognizes an EGFR peptide is selected from the group consisting of Seq. Id. No. 1, Seq. Id. No. 2, Seq. Id. No. 3, Seq. Id. No. 4, Seq. Id. No. 5, Seq. Id. No. 6, Seq. Id. No. 7, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 15, Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 64, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 102, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 309, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 370, Seq. Id. No. 371, Seq. Id. No. 372, Seq. Id. No. 373, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516, Seq. Id. No. 517, Seq. Id. No. 518, Seq. Id. No. 519, Seq. Id. No. 520, Seq. Id. No. 521, Seq. Id. No. 522, Seq. Id. No. 523, Seq. Id. No. 524, Seq. Id. No. 525, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 556, Seq. Id. No. 557, Seq. Id. No. 558, Seq. Id. No. 559, Seq. Id. No. 560, Seq. Id. No. 561, Seq. Id. No. 562, Seq. Id. No. 563, Seq. Id. No. 564, Seq. Id. No. 565, Seq. Id. No. 566, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599, Seq. Id. No. 600, Seq. Id. No. 601, Seq. Id. No. 602, Seq. Id. No. 603, Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618, and Seq. Id. No. 619.

20. The method according to any of claims 19, wherein the autoantibody recognizes an EGFR peptide that is selected from the group consisting of Seq. Id. No. 3, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 64, Seq. Id. No. 102, Seq. Id. No. 309, Seq. Id. No. 370, Seq. Id. No. 373, Seq. Id. No. 519, Seq. Id. No. 520, Seq. Id. No. 521, Seq. Id. No. 525, Seq. Id. No. 556, Seq. Id. No. 558, Seq. Id. No. 561, Seq. Id. No. 566, Seq. Id. No. 600, Seq. Id. No. 602 and Seq. Id. No. 603.

21. The method according to any of claims 17-20, wherein the level of an autoantibody in the blood sample of the human subject is 5 times higher than the level of said autoantibody representative for a human subject of a healthy population.

22. Erlotinib or a pharmaceutically acceptable salt thereof, in particular erlotinib hydrochloride, for use in treating a NSCLC patient identified by a method of any of claims 17 to 21 comprising administering erlotinib or a pharmaceutically acceptable salt thereof, in particular erlotinib hydrochloride to the patient.

23. Use of an autoantibody for predicting the response of a NSCLC patient to erlotinib or a pharmaceutically acceptable salt thereof, in particular erlotinib hydrochloride, treatment, which antibody was identified by a method of any of claims 17 to 21.

24. A kit for detecting in a blood sample of the human subject a level of one or more autoantibodies selected from the group of autoantibodies recognizing human EGFR, wherein an increased level of said autoantibodies selected from the group of autoantibodies recognizing human EGFR in the blood sample of the human subject compared to a level of said autoantibodies representative for a human subject of a healthy population is indicative for non-small cell lung cancer.

25. A kit according to claim 24, wherein the autoantibody recognizes an EGFR peptide that is selected from the group consisting of Seq. Id. No. 3, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 64, Seq. Id. No. 102, Seq. Id. No. 309, Seq. Id. No. 370, Seq. Id. No. 373, Seq. Id. No. 519, Seq. Id. No. 520, Seq. Id. No. 521, Seq. Id. No. 525, Seq. Id. No. 556, Seq. Id. No. 558, Seq. Id. No. 561, Seq. Id. No. 566, Seq. Id. No. 600, Seq. Id. No. 602 and Seq. Id. No. 603.

26. A kit according to claim 25, wherein the autoantibody recognizes an EGFR peptide that is selected from the group consisting of Seq. Id. No. 519, Seq. Id. No. 520, Seq. Id. No. 521, and Seq. Id. No. 561.

27. A method of diagnosis of non-small cell lung cancer in a human subject comprising:
a) measuring in a blood sample of the human subject a level of an autoantibody selected from the group of autoantibodies recognizing mutated human EGFR,
b) comparing the level of said autoantibody to a reference level, and
c) providing a diagnosis of non-small cell lung cancer when the level of said autoantibody is above the reference level.

28. A method of diagnosis of non-small cell lung cancer in a human subject comprising:
a) measuring in a blood sample of the human subject a level of an autoantibody selected from the group of autoantibodies recognizing mutated human EGFR,
b) comparing the level of said autoantibody to a reference level, and
c) recommending a treatment when the level of said autoantibody is above the reference level.

29. The method according to claim 27 or 28, wherein the autoantibody recognizes a mutated EGFR peptide is selected from the group consisting of Seq. Id. No. 554, Seq. Id. No. 555, Seq. Id. No. 556, Seq. Id. No. 557, Seq. Id. No. 558, Seq. Id. No. 559, and Seq. Id. No. 560.

30. The method according to any of claims 28-29, wherein the autoantibody recognizes a mutated EGFR peptide is selected from the group consisting of Seq. Id. No. 1, Seq. Id. No. 2, Seq. Id. No. 4, Seq. Id. No. 5, Seq. Id. No. 6, Seq. Id. No. 7, Seq. Id. No. 15, Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516, Seq. Id. No. 517, Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599, Seq. Id. No. 601, Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

31. The method according to any of claims 27-30, wherein the autoantibody recognizes a mutated EGFR peptide that is selected from the groups consisting of Seq. Id. No. 1, Seq. Id. No. 2, Seq. Id. No. 4, Seq. Id. No. 5, Seq. Id. No. 6, Seq. Id. No. 7 and Seq. Id. No. 15.

32. The method according to any of claims 27-30, wherein the autoantibody recognizes a mutated EGFR peptide that is selected from the group consisting of Seq. Id. No. 16, Seq. Id. No. 17, Seq. Id. No. 18, Seq. Id. No. 19, Seq. Id. No. 20, Seq. Id. No. 21, Seq. Id. No. 22, Seq. Id. No. 23, Seq. Id. No. 24, Seq. Id. No. 25, Seq. Id. No. 26, Seq. Id. No. 27, Seq. Id. No. 28, Seq. Id. No. 29, Seq. Id. No. 30, Seq. Id. No. 31, Seq. Id. No. 32, Seq. Id. No. 33, Seq. Id. No. 34, Seq. Id. No. 35, Seq. Id. No. 36, Seq. Id. No. 37, Seq. Id. No. 38, Seq. Id. No. 39, Seq. Id. No. 40, Seq. Id. No. 41, Seq. Id. No. 42, Seq. Id. No. 43, Seq. Id. No. 44, Seq. Id. No. 45, Seq. Id. No. 46, Seq. Id. No. 47, Seq. Id. No. 48, Seq. Id. No. 49, Seq. Id. No. 50, Seq. Id. No. 51, Seq. Id. No. 52, Seq. Id. No. 53, Seq. Id. No. 54, Seq. Id. No. 55, Seq. Id. No. 56, Seq. Id. No. 57, Seq. Id. No. 58, Seq. Id. No. 59, Seq. Id. No. 60, Seq. Id. No. 61, Seq. Id. No. 62, Seq. Id. No. 63, Seq. Id. No. 65, Seq. Id. No. 66, Seq. Id. No. 67, Seq. Id. No. 68, Seq. Id. No. 69, Seq. Id. No. 70, Seq. Id. No. 71, Seq. Id. No. 72, Seq. Id. No. 73, Seq. Id. No. 74, Seq. Id. No. 75, Seq. Id. No. 76, Seq. Id. No. 77, Seq. Id. No. 78, Seq. Id. No. 79, Seq. Id. No. 80, Seq. Id. No. 81, Seq. Id. No. 82, Seq. Id. No. 83, Seq. Id. No. 84, Seq. Id. No. 85, Seq. Id. No. 86, Seq. Id. No. 87, Seq. Id. No. 88, Seq. Id. No. 89, Seq. Id. No. 90, Seq. Id. No. 91, Seq. Id. No. 92, Seq. Id. No. 93, Seq. Id. No. 94, Seq. Id. No. 95, Seq. Id. No. 96, Seq. Id. No. 97, Seq. Id. No. 98, Seq. Id. No. 99, Seq. Id. No. 100, Seq. Id. No. 101, Seq. Id. No. 103, Seq. Id. No. 104, Seq. Id. No. 105, Seq. Id. No. 106, Seq. Id. No. 107, Seq. Id. No. 108, Seq. Id. No. 109, Seq. Id. No. 110, Seq. Id. No. 111, Seq. Id. No. 112, Seq. Id. No. 113, Seq. Id. No. 114, Seq. Id. No. 115, Seq. Id. No. 116, Seq. Id. No. 117, Seq. Id. No. 118, Seq. Id. No. 119, Seq. Id. No. 120, Seq. Id. No. 121, Seq. Id. No. 122, Seq. Id. No. 123, Seq. Id. No. 124, Seq. Id. No. 125, Seq. Id. No. 126, Seq. Id. No. 127, Seq. Id. No. 128, Seq. Id. No. 129, Seq. Id. No. 130, Seq. Id. No. 131, Seq. Id. No. 132, Seq. Id. No. 133, Seq. Id. No. 134, Seq. Id. No. 135, Seq. Id. No. 136, Seq. Id. No. 137, Seq. Id. No. 138, Seq. Id. No. 139, Seq. Id. No. 140, Seq. Id. No. 141, Seq. Id. No. 142, Seq. Id. No. 143, Seq. Id. No. 144, Seq. Id. No. 145, Seq. Id. No. 146, Seq. Id. No. 147, Seq. Id. No. 148, Seq. Id. No. 149, Seq. Id. No. 150, Seq. Id. No. 151, Seq. Id. No. 152, Seq. Id. No. 153, Seq. Id. No. 154, Seq. Id. No. 155, Seq. Id. No. 156, Seq. Id. No. 157, Seq. Id. No. 158, Seq. Id. No. 159, Seq. Id. No. 160, Seq. Id. No. 161, Seq. Id. No. 162, Seq. Id. No. 163, Seq. Id. No. 164, Seq. Id. No. 165, Seq. Id. No. 166, Seq. Id. No. 167, Seq. Id. No. 168, Seq. Id. No. 169, Seq. Id. No. 170, Seq. Id. No. 171, Seq. Id. No. 172, Seq. Id. No. 173, Seq. Id. No. 174, Seq. Id. No. 175, Seq. Id. No. 176, Seq. Id. No. 177, Seq. Id. No. 178, Seq. Id. No. 179, Seq. Id. No. 180, Seq. Id. No. 181, Seq. Id. No. 182, Seq. Id. No. 183, Seq. Id. No. 184, Seq. Id. No. 185, Seq. Id. No. 186, Seq. Id. No. 187, Seq. Id. No. 188, Seq. Id. No. 189, Seq. Id. No. 190, Seq. Id. No. 191, Seq. Id. No. 192, Seq. Id. No. 193, Seq. Id. No. 194, Seq. Id. No. 195, Seq. Id. No. 196, Seq. Id. No. 197, Seq. Id. No. 198, Seq. Id. No. 199, Seq. Id. No. 200, Seq. Id. No. 201, Seq. Id. No. 202, Seq. Id. No. 203, Seq. Id. No. 204, Seq. Id. No. 205, Seq. Id. No. 206, Seq. Id. No. 207, Seq. Id. No. 208, Seq. Id. No. 209, Seq. Id. No. 210, Seq. Id. No. 211, Seq. Id. No. 212, Seq. Id. No. 213, Seq. Id. No. 214, Seq. Id. No. 215, Seq. Id. No. 216, Seq. Id. No. 217, Seq. Id. No. 218, Seq. Id. No. 219, Seq. Id. No. 220, Seq. Id. No. 221, Seq. Id. No. 222, Seq. Id. No. 223, Seq. Id. No. 224, Seq. Id. No. 225, Seq. Id. No. 226, Seq. Id. No. 227, Seq. Id. No. 228, Seq. Id. No. 229, Seq. Id. No. 230, Seq. Id. No. 231, Seq. Id. No. 232, Seq. Id. No. 233, Seq. Id. No. 234, Seq. Id. No. 235, Seq. Id. No. 236, Seq. Id. No. 237, Seq. Id. No. 238, Seq. Id. No. 239, Seq. Id. No. 240, Seq. Id. No. 241, Seq. Id. No. 242, Seq. Id. No. 243, Seq. Id. No. 244, Seq. Id. No. 245, Seq. Id. No. 246, Seq. Id. No. 247, Seq. Id. No. 248, Seq. Id. No. 249, Seq. Id. No. 250, Seq. Id. No. 251, Seq. Id. No. 252, Seq. Id. No. 253, Seq. Id. No. 254, Seq. Id. No. 255, Seq. Id. No. 256, Seq. Id. No. 257, Seq. Id. No. 258, Seq. Id. No. 259, Seq. Id. No. 260, Seq. Id. No. 261, Seq. Id. No. 262, Seq. Id. No. 263, Seq. Id. No. 264, Seq. Id. No. 265, Seq. Id. No. 266, Seq. Id. No. 267, Seq. Id. No. 268, Seq. Id. No. 269, Seq. Id. No. 270, Seq. Id. No. 271, Seq. Id. No. 272, Seq. Id. No. 273, Seq. Id. No. 274, Seq. Id. No. 275, Seq. Id. No. 276, Seq. Id. No. 277, Seq. Id. No. 278, Seq. Id. No. 279, Seq. Id. No. 280, Seq. Id. No. 281, Seq. Id. No. 282, Seq. Id. No. 283, Seq. Id. No. 284, Seq. Id. No. 285, Seq. Id. No. 286, Seq. Id. No. 287, Seq. Id. No. 288, Seq. Id. No. 289, Seq. Id. No. 290, Seq. Id. No. 291, Seq. Id. No. 292, Seq. Id. No. 293, Seq. Id. No. 294, Seq. Id. No. 295, Seq. Id. No. 296, Seq. Id. No. 297, Seq. Id. No. 298, Seq. Id. No. 299, Seq. Id. No. 300, Seq. Id. No. 301, Seq. Id. No. 302, Seq. Id. No. 303, Seq. Id. No. 304, Seq. Id. No. 305, Seq. Id. No. 306, Seq. Id. No. 307, Seq. Id. No. 308, Seq. Id. No. 310, Seq. Id. No. 311, Seq. Id. No. 312, Seq. Id. No. 313, Seq. Id. No. 314, Seq. Id. No. 315, Seq. Id. No. 316, Seq. Id. No. 317, Seq. Id. No. 318, Seq. Id. No. 319, Seq. Id. No. 320, Seq. Id. No. 321, Seq. Id. No. 322, Seq. Id. No. 323, Seq. Id. No. 324, Seq. Id. No. 325, Seq. Id. No. 326, Seq. Id. No. 327, Seq. Id. No. 328, Seq. Id. No. 329, Seq. Id. No. 330, Seq. Id. No. 331, Seq. Id. No. 332, Seq. Id. No. 333, Seq. Id. No. 334, Seq. Id. No. 335, Seq. Id. No. 336, Seq. Id. No. 337, Seq. Id. No. 338, Seq. Id. No. 339, Seq. Id. No. 340, Seq. Id. No. 341, Seq. Id. No. 342, Seq. Id. No. 343, Seq. Id. No. 344, Seq. Id. No. 345, Seq. Id. No. 346, Seq. Id. No. 347, Seq. Id. No. 348, Seq. Id. No. 349, Seq. Id. No. 350, Seq. Id. No. 351, Seq. Id. No. 352, Seq. Id. No. 353, Seq. Id. No. 354, Seq. Id. No. 355, Seq. Id. No. 356, Seq. Id. No. 357, Seq. Id. No. 358, Seq. Id. No. 359, Seq. Id. No. 360, Seq. Id. No. 361, Seq. Id. No. 362, Seq. Id. No. 363, Seq. Id. No. 364, Seq. Id. No. 365, Seq. Id. No. 366, Seq. Id. No. 367, Seq. Id. No. 368, Seq. Id. No. 369, Seq. Id. No. 371, Seq. Id. No. 372, Seq. Id. No. 374, Seq. Id. No. 375, Seq. Id. No. 376, Seq. Id. No. 377, Seq. Id. No. 378, Seq. Id. No. 379, Seq. Id. No. 380, Seq. Id. No. 381, Seq. Id. No. 382, Seq. Id. No. 383, Seq. Id. No. 384, Seq. Id. No. 385, Seq. Id. No. 386, Seq. Id. No. 387, Seq. Id. No. 388, Seq. Id. No. 389, Seq. Id. No. 390, Seq. Id. No. 391, Seq. Id. No. 392, Seq. Id. No. 393, Seq. Id. No. 394, Seq. Id. No. 395, Seq. Id. No. 396, Seq. Id. No. 397, Seq. Id. No. 398, Seq. Id. No. 399, Seq. Id. No. 400, Seq. Id. No. 401, Seq. Id. No. 402, Seq. Id. No. 403, Seq. Id. No. 404, Seq. Id. No. 405, Seq. Id. No. 406, Seq. Id. No. 407, Seq. Id. No. 408, Seq. Id. No. 409, Seq. Id. No. 410, Seq. Id. No. 411, Seq. Id. No. 412, Seq. Id. No. 413, Seq. Id. No. 414, Seq. Id. No. 415, Seq. Id. No. 416, Seq. Id. No. 417, Seq. Id. No. 418, Seq. Id. No. 419, Seq. Id. No. 420, Seq. Id. No. 421, Seq. Id. No. 422, Seq. Id. No. 423, Seq. Id. No. 424, Seq. Id. No. 425, Seq. Id. No. 426, Seq. Id. No. 427, Seq. Id. No. 428, Seq. Id. No. 429, Seq. Id. No. 430, Seq. Id. No. 431, Seq. Id. No. 432, Seq. Id. No. 433, Seq. Id. No. 434, Seq. Id. No. 435, Seq. Id. No. 436, Seq. Id. No. 437, Seq. Id. No. 438, Seq. Id. No. 439, Seq. Id. No. 440, Seq. Id. No. 441, Seq. Id. No. 442, Seq. Id. No. 443, Seq. Id. No. 444, Seq. Id. No. 445, Seq. Id. No. 446, Seq. Id. No. 447, Seq. Id. No. 448, Seq. Id. No. 449, Seq. Id. No. 450, Seq. Id. No. 451, Seq. Id. No. 452, Seq. Id. No. 453, Seq. Id. No. 454, Seq. Id. No. 455, Seq. Id. No. 456, Seq. Id. No. 457, Seq. Id. No. 458, Seq. Id. No. 459, Seq. Id. No. 460, Seq. Id. No. 461, Seq. Id. No. 462, Seq. Id. No. 463, Seq. Id. No. 464, Seq. Id. No. 465, Seq. Id. No. 466, Seq. Id. No. 467, Seq. Id. No. 468, Seq. Id. No. 469, Seq. Id. No. 470, Seq. Id. No. 471, Seq. Id. No. 472, Seq. Id. No. 473, Seq. Id. No. 474, Seq. Id. No. 475, Seq. Id. No. 476, Seq. Id. No. 477, Seq. Id. No. 478, Seq. Id. No. 479, Seq. Id. No. 480, Seq. Id. No. 481, Seq. Id. No. 482, Seq. Id. No. 483, Seq. Id. No. 484, Seq. Id. No. 485, Seq. Id. No. 486, Seq. Id. No. 487, Seq. Id. No. 488, Seq. Id. No. 489, Seq. Id. No. 490, Seq. Id. No. 491, Seq. Id. No. 492, Seq. Id. No. 493, Seq. Id. No. 494, Seq. Id. No. 495, Seq. Id. No. 496, Seq. Id. No. 497, Seq. Id. No. 498, Seq. Id. No. 499, Seq. Id. No. 500, Seq. Id. No. 501, Seq. Id. No. 502, Seq. Id. No. 503, Seq. Id. No. 504, Seq. Id. No. 505, Seq. Id. No. 506, Seq. Id. No. 507, Seq. Id. No. 508, Seq. Id. No. 509, Seq. Id. No. 510, Seq. Id. No. 511, Seq. Id. No. 512, Seq. Id. No. 513, Seq. Id. No. 514, Seq. Id. No. 515, Seq. Id. No. 516 and Seq. Id. No. 517.

33. The method according to any of claims 27-30, wherein the autoantibody recognizes a mutated EGFR peptide that is selected from the group consisting of Seq. Id. No. 518, Seq. Id. No. 522, Seq. Id. No. 523, Seq. Id. No. 524, Seq. Id. No. 526, Seq. Id. No. 527, Seq. Id. No. 528, Seq. Id. No. 529, Seq. Id. No. 530, Seq. Id. No. 531, Seq. Id. No. 532, Seq. Id. No. 533, Seq. Id. No. 534, Seq. Id. No. 535, Seq. Id. No. 536, Seq. Id. No. 537, Seq. Id. No. 538, Seq. Id. No. 539, Seq. Id. No. 540, Seq. Id. No. 541, Seq. Id. No. 542, Seq. Id. No. 543, Seq. Id. No. 544, Seq. Id. No. 545, Seq. Id. No. 546, Seq. Id. No. 547, Seq. Id. No. 548, Seq. Id. No. 549, Seq. Id. No. 550, Seq. Id. No. 551, Seq. Id. No. 552, Seq. Id. No. 553, Seq. Id. No. 554, Seq. Id. No. 555, Seq. Id. No. 557, Seq. Id. No. 559, Seq. Id. No. 560, Seq. Id. No. 562, Seq. Id. No. 563, Seq. Id. No. 564, Seq. Id. No. 565, Seq. Id. No. 567, Seq. Id. No. 568, Seq. Id. No. 569, Seq. Id. No. 570, Seq. Id. No. 571, Seq. Id. No. 572, Seq. Id. No. 573, Seq. Id. No. 574, Seq. Id. No. 575, Seq. Id. No. 576, Seq. Id. No. 577, Seq. Id. No. 578, Seq. Id. No. 579, Seq. Id. No. 580, Seq. Id. No. 581, Seq. Id. No. 582, Seq. Id. No. 583, Seq. Id. No. 584, Seq. Id. No. 585, Seq. Id. No. 586, Seq. Id. No. 587, Seq. Id. No. 588, Seq. Id. No. 589, Seq. Id. No. 590, Seq. Id. No. 591, Seq. Id. No. 592, Seq. Id. No. 593, Seq. Id. No. 594, Seq. Id. No. 595, Seq. Id. No. 596, Seq. Id. No. 597, Seq. Id. No. 598, Seq. Id. No. 599 and Seq. Id. No. 601.

34. The method according to any of claims 27-30, wherein the autoantibody recognizes a mutated EGFR peptide that is selected from the group consisting of Seq. Id. No. 604, Seq. Id. No. 605, Seq. Id. No. 606, Seq. Id. No. 607, Seq. Id. No. 608, Seq. Id. No. 609, Seq. Id. No. 610, Seq. Id. No. 611, Seq. Id. No. 612, Seq. Id. No. 613, Seq. Id. No. 614, Seq. Id. No. 615, Seq. Id. No. 616, Seq. Id. No. 617, Seq. Id. No. 618 and Seq. Id. No. 619.

35. The method according to any of claims 27-34, wherein the level of an autoantibody in the blood sample of the human subject is 5 times higher than the level of said autoantibody representative for a human subject of a healthy population.

36. A method of diagnosis of non-small cell lung cancer in a human subject comprising:
a) measuring in a blood sample of the human subject a level of an autoantibody selected from the group of autoantibodies recognizing human EGFR,
b) comparing the level of said autoantibody to a reference level, and
c) providing a diagnosis of non-small cell lung cancer when the level of said autoantibody is above the reference level.

37. A method of diagnosis of non-small cell lung cancer in a human subject comprising:
a) measuring in a blood sample of the human subject a level of an autoantibody selected from the group of autoantibodies recognizing human EGFR,
b) comparing the level of said autoantibody to a reference level, and
c) recommending a treatment when the level of said autoantibody is above the reference level.

38. The method according to claim 36 or 37, wherein the autoantibody recognizes an EGFR peptide that is selected from the group consisting of Seq. Id. No. 3, Seq. Id. No. 8, Seq. Id. No. 9, Seq. Id. No. 10, Seq. Id. No. 11, Seq. Id. No. 12, Seq. Id. No. 13, Seq. Id. No. 14, Seq. Id. No. 64, Seq. Id. No. 102, Seq. Id. No. 309, Seq. Id. No. 370, Seq. Id. No. 373, Seq. Id. No. 519, Seq. Id. No. 520, Seq. Id. No. 521, Seq. Id. No. 525, Seq. Id. No. 556, Seq. Id. No. 558, Seq. Id. No. 561, Seq. Id. No. 566, Seq. Id. No. 600, Seq. Id. No. 602 and Seq. Id. No. 603.

39. The method according to any of claims 37-49, wherein the level of an autoantibody in the blood sample of the human subject is 5 times higher than the level of said autoantibody representative for a human subject of a healthy population.

40. The method according to any of claims 28-35, 37-39, wherein the treatment is erlotinib.
